(19) United States
(12) Patent Application Publication
(10) Pub. No.: US 2009/0111109 A1
(43) Pub. Date: Apr. 30, 2009

(54) Genes relating to gastric cancer metastasis

(76) Inventors: Tsung-Fan Tuan, Zhunan Town (TW); Heng-Liang Lin, Zhunan Town (TW); Chiung-Tong Chen, Zhunan Town (TW)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/230,060

(22) Filed: Aug. 22, 2008

Related U.S. Application Data
(60) Provisional application No. 60/966,074, filed on Aug. 24, 2007.

(51) Int. Cl. C12Q 1/68; G01N 33/53; C40B 30/04; C12N 5/08

(52) U.S. Cl. 435/6; 435/7.1; 506/9; 435/366

(57) ABSTRACT

The disclosure provides polynucleotides and polypeptides associated with gastric cancer cells, particularly those having a tendency to metastasize. Also provided are methods and kits for detecting, diagnosing, and/or monitoring metastatic gastric cancer cells.

### CROSS-REFERENCE TO RELATED PATENT APPLICATION

This application claims the benefit, pursuant to 35 U.S.C. §119(e), of U.S. provisional patent application No. 60/966,074, filed Aug. 24, 2007, which is incorporated herein by reference in its entirety.

### DESCRIPTION OF THE INVENTION

1. Field of Invention

The present disclosure relates to gastric cancer diagnosis and treatments, and specifically to the identification of genes and their encoded polypeptides related to gastric cancer metastasis and the use of the identified genes and polypeptides as markers in detecting, diagnosing, prognosing, and/or monitoring subjects with cancer, in particular metastatic gastric cancers.

2. Background of the Invention

Gastric cancer is a serious health problem and remains the second most common type of fatal cancer worldwide. Mainly, there are two histologic types of gastric cancers: (1) well-differentiated or intestinal type, and (2) undifferentiated or diffuse type (Best Pract. Res. Clin. Gastroenterol, 2006 20(4):651-674). Intestinal type tumors predominate in high risk geographic areas, such as East Asia, Eastern Europe, Central and South America, whereas diffusion type tumors have a more uniform geographic distribution (World J Gastroenterol, 2006 12(3):354-362). According to a recent survey, the morbidity and mortality of gastric cancer are higher in Taiwan than that in the US (CA Cancer J. Clin., 2006 56:106-130; J. Formos. Med. Assoc., 2004 103:171-185).

Over the years, large-scale gene expression studies with array-based hybridization and serial analysis of gene expression (SAGE) have been performed and several genes have been identified (Oncology 2005 69:17-22). These findings may provide knowledge and tools for diagnosis and treatment of gastric cancers in the early stage, thereby ensuring excellent survival rate for patients with early stage gastric cancers. However, once cancer cells start to migrate or metastasize, prognosis and treatment become difficult. For humans, when gastric cancer metastasizes to liver and peritoneum, the 5-year-survival rate drops to 10.1% (Cancer Cell, 2004 5:121-125). Therefore, there exists a need to identify metastatic gastric cancer-associated genes that are useful as markers for detecting, diagnosing, prognosing, and/or monitoring gastric cancers, especially for those having a tendency to metastasize.

### SUMMARY OF THE INVENTION

This disclosure provides methods of detecting metastatic gastric cancer comprising: a) obtaining one or more tissue samples from a subject; b) measuring in the tissue samples the levels of one or more polynucleotides comprising nucleotide sequences at least 90% identical to nucleotide sequences chosen from SEQ ID NOs: 37-53; c) determining whether the expression levels of the one or more polynucleotides are lower than the expression levels of the one or more polynucleotides in normal tissue samples, wherein a lower expression level indicates the presence of metastatic gastric cancer.

In one embodiment, the one or more polynucleotides comprise nucleotide sequences chosen from SEQ ID NOs: 37-53. In another embodiment, the expression levels of the one or more polynucleotides are determined by a method chosen from: a) hybridizing one or more probes to the one or more polynucleotides and measuring the amount of the one or more probes bound to the one or more polynucleotides; b) amplifying the polynucleotides using PCR and measuring the levels of the PCR products; c) Serial Analysis of Gene Expression (SAGE); and d) Massively Parallel Signature Sequencing (MPSS). In another embodiment, an expression level of the one or more polynucleotides in the sample is at least three-fold lower than a level of the one or more polynucleotides in normal tissue samples. In another embodiment, the tissue sample is chosen from primary gastric cancer tissue, metastatic gastric cancer tissue, and body fluid of the subject. In another embodiment, the body fluid is chosen from blood, plasma, serum, peritoneal fluid, urine, and saliva.

The disclosure provides a method of detecting metastatic gastric cancer comprising: a) obtaining one or more tissue samples from a subject; b) measuring in the tissue samples the expression levels of one or more polypeptides comprising an amino acid sequence at least 90% identical to amino acid sequences chosen from SEQ ID NOs: 58-73; c) determining whether the expression levels of the one or more polypeptides in the are lower than the expression levels of the one or more polypeptides in normal tissue samples, wherein a lower expression level indicates the presence of metastatic gastric cancer cells.

In one embodiment, the one or more polypeptides comprise amino acid sequences chosen from SEQ ID NOs: 58-73. In another embodiment, the levels of the one or more polypeptides are determined by a method chosen from: a) contacting the one or more polypeptides with one or more antibodies and detecting one or more complexes comprising the one or more polypeptides and the one or more antibodies; b) mass-spectrometry; c) hybridization to a protein array. In another embodiment, the tissue sample is chosen from primary gastric cancer tissue, metastatic gastric cancer tissue, and body fluid of the subject. In another embodiment, the body fluid is chosen from blood, plasma, serum, peritoneal fluid, urine, and saliva.

The disclosure provides a method of detecting metastatic gastric cancer comprising: a) obtaining one or more tissue samples from a subject; b) measuring in the tissue samples the expression levels of one or more polynucleotides, wherein the one or more polynucleotides comprise nucleotide sequences at least 90% identical to nucleotide sequences chosen from SEQ ID NOs: 54-57; c) determining whether the expression levels of the one or more polynucleotides in the tissue samples is higher than the expression levels of the one or more polynucleotides in normal tissue samples, wherein a higher expression level indicates the presence of metastatic gastric cancer cells.

In one embodiment, the one or more polynucleotides comprise nucleotide sequences chosen from SEQ ID NOs: 54-57. In another embodiment, the expression levels of the one or more polynucleotides are determined by a method chosen from: a) hybridizing one or more probes to the one or more polynucleotides and measuring the amount of the one or more probes bound to the one or more polynucleotides; b) amplifying the polynucleotides using PCR and measuring the levels of the PCR products; c) Serial Analysis of Gene Expression (SAGE); and d) Massively Parallel Signature Sequencing (MPSS). In another embodiment, an expression level of the one or more polynucleotides in the sample is at least three-fold higher than a level of the one or more polynucleotides in normal tissue samples.

The disclosure provides a method of detecting metastatic gastric cancer comprising: a) obtaining one or more tissue samples from a subject; b) measuring in the tissue samples the expression levels of one or more polypeptides, comprising polypeptide sequences at least 90% identical to polypeptide sequences chosen from SEQ ID NOs: 74-77; c) determining whether the expression levels of the one or more polypeptides in the tissue samples are higher than the expression levels of the one or more polypeptides in normal tissue samples, wherein a higher expression level indicates the presence of metastatic gastric cancer cells.

In one embodiment, the one or more polypeptides comprise amino acid sequences chosen from SEQ ID NOs: 74-77. In another embodiment, the levels of the one or more polypeptides are determined by a method chosen from: a) contacting the one or more polypeptides with one or more antibodies and detecting one or more complexes comprising the one or more polypeptides and the one or more antibodies; b) mass-spectrometry; c) hybridization to a protein array. In another embodiment, the tissue sample is chosen from primary gastric cancer tissue, metastatic gastric cancer tissue, and body fluid of the subject. In another embodiment, the body fluid is chosen from blood, plasma, serum, peritoneal fluid, urine, and saliva.

The disclosure provides a kit comprising a composition chosen from: a) one or more polynucleotides complementary to nucleotide sequences at least 90% identical to nucleotide sequences comprising SEQ ID NOs: 37-57; and b) one more antibodies specific for a polypeptide chosen from polypeptides at least 90% identical to polypeptide sequences comprising SEQ ID NOs: 58-77.

The disclosure provides a tumor cell line chosen from MKN45-GFP TW4, as deposited with ______ under the accession number ______ on ______, MKN45-GFP TW5, as deposited with ______ under the accession number ______ on ______, MKN45-GFP TW8, as deposited with ______ under the accession number ______ on ______, MKN45-GFP TW10, as deposited with ______ under the accession number ______ on ______, and MKN45-GFP TW12, as deposited with ______ under the accession number ______ on ______.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows photographs of MKN45-GFP cells viewed under an inverted microscope with either direct light (A) or UV light (B).

FIG. 2A illustrates in vitro invasion ability of MKN45 sublines. The upper panel shows the image of each MKN45 subline after staining with hematoxylin, and the lower panel shows the cell density of the lower sides of a TRANSWELL® membrane for each MKN45 subline.

FIG. 2B illustrates the proliferation rate of each of the MKN45 sublines determined by MTS/PMS assay.

FIG. 3A illustrates the hierarchical clustering analysis of five hundred and twenty-five (525) microarray spots, in which three hundred and thirty-five (335) descending spots and one hundred ninety (190) ascending spots were identified.

FIG. 3B shows three groups of ascending and descending expression profiles from eighteen groups.

FIG. 4 illustrates the grouping of invasion/metastasis-related genes, including (A) angiogenesis-related genes, (B) cell cycle regulators, (C) cytoskeleton and motility molecules, (D) protease and adhesion proteins, and (E) signal transduction molecules.

FIG. 5A illustrates the PCR analysis of the RNA expression profile of the genes of the disclosure.

FIG. 5B illustrates the Western blot analysis of a protein expression profile of the proteins of the disclosure.

### DETAILED DESCRIPTION OF THE INVENTION

The disclosure relates to the identification of novel genes that are expressed in highly metastatic gastric cancer cell lines, as well as the use of the identified genes as markers for detecting, diagnosing, and/or monitoring gastric cancer cells.

Cell invasion is exhibited by both normal cells in responses such as inflammation and by tumor cells in the process of metastasis. Invasive gastric cancer cell lines were established using a well-known cell migration, chemotaxis, and invasion assay protocol, as described below. This protocol allows selection of invasive cells through the use of chemoattractants such as serum or growth factors. Cells that respond to chemoattractants and move from one culture well across a permeable support to a receiver well are categorized as invasive cells, or cells that metastasize.

In one embodiment, several invasive gastric cancer cell sublines, particularly MKN45 sublines, were chosen by an in vitro cell invasion assay in a commercial TRANSWELL® Plate (Corning, Acton, Mass.). MKN45 cells that went through 4, 8,10 and 12 selection cycles were termed as MKN45 TW4, MKN45 TW8, MKN45 TW10 and MKN45 TW12 cells, respectively. Each of the selected sublines exhibited high invasive ability and similar proliferation rate with little differences in doubling time. These invasive sublines are therefore preferred targets for the selection of metastatic gastric cancer genes.

Taking advantage of the selected MKN45 sublines with high invasive potential and microarray technology, metastatic gastric cancer genes were identified in the highly metastatic cell lines.

76 metastatic genes were identified using an RNA microarray, with at least 3-fold higher or lower expression level in metastatic cancer cells than in normal cells. Among these 76 genes, 22 genes are novel and have never been disclosed or suggested in the prior art as being related to gastric cancer metastasis. The novel genes' polynucleotide sequences and the corresponding protein polypeptide sequences are disclosed herein.

This disclosure thus provides isolated polynucleotides relating to gastric cancer cell metastasis, comprising nucleotide sequences at least 90%, 91%, 92%, 93%, 94%, 95%, 96%, 97%, 98%, or 99% identical to nucleotide sequences selected from SEQ ID NOs: 37-57.

This disclosure provides purified polypeptides comprising amino acid sequences at least 85%, 86%, 87%, 88%, 89%, 90%, 91%, 92%, 93%, 94%, 95%, 96%, 97%, 98%, or 99% identical to amino acid sequences selected from SEQ ID NO: 58-77 or a fragment thereof.

The detection and diagnosis of metastatic gastric cancer cells can be carried out by monitoring the expression levels of the identified genes. This disclosure thus provides a method of detecting, diagnosing, and/or monitoring metastatic gastric cancer cells in a tissue sample, comprising measuring the expression level in the tissue sample of an expressed product of a gene having a nucleotide sequence at least 90%, 91%, 92%, 93%, 94%, 95%, 96%, 97%, 98%, or 99% identical to a nucleotide sequence selected from SEQ ID NOs: 37-57. A tissue sample that expresses the gene product of SEQ ID NOs: 37-53 at least three-fold lower than that of a control sample, or a normal tissue sample, is categorized as being metastatic. On the other hand, a tissue sample that expresses the gene product of SEQ ID NOs: 54-57 at least three-fold higher than that of a control sample (a normal tissue sample) is also categorized as being metastatic. The expression level of the expressed gene product may be determined by examining the level of mRNA corresponding to the gene, or the level of protein encoded by the gene, using methods known in the art. The tissue samples may be primary gastric cancer tissue, metastatic gastric cancer tissue, or body fluid such as blood, plasma, serum, urine, saliva, peritoneal fluid, or any other body-secretion of a subject suspected of having gastric cancer. Tissue samples may be obtained using standard methods.

In addition to the isolated polynucleotides or purified polypeptides such as those described above, the invention further features one or more antibodies against one or more polypeptides or a fragment thereof encoded by amino acid sequences selected from SEQ ID NOs: 58-77.

As described above, lower levels of expression products of any of SEQ ID NOs: 37-53 or higher levels of expression products of any of SEQ ID NOs: 54-57 indicate the presence of metastatic gastric cancer cells. A kit may thus be developed for detecting or diagnosing metastatic gastric cancer cells based on hybridization assay, Western blot, or ELISA and other methods known in the art. The kit may contain, in separate containers, one or more probes comprising one or more nucleotide sequences that are complementary to mRNAs of genes comprising nucleotide sequences at least 90%, 91%, 92%, 93%, 94%, 95%, 96%, 97%, 98%, or 99% identical to nucleotide sequences selected from SEQ ID NOs: 37-57. Alternatively, the kit may contain antibodies against one or more polypeptides or a fragment thereof encoded by amino acid sequences selected from SEQ ID NOs: 58-77. The kit may further comprise probes or antibodies; control formulations (either positive or negative) and/or a detectable label; and instructions such as written direction, audio-tape, VCR, CD-ROM, other electronic formats, and etc. for carrying out the assay included in the kit.

In another embodiment, this invention provides a method of detecting, diagnosing, and/or monitoring metastatic gastric cancer cells in one or more tissue samples, comprising measuring in the tissue samples the expression level of one or more polynucleotides comprising nucleotide sequences at least 90%, 91%, 92%, 93%, 94%, 95%, 96%, 97%, 98%, or 99% identical to nucleotide sequences selected from SEQ ID NOs: 37-57. A tissue sample that expresses the polynucleotides at a higher or lower level than that of a control sample, or a normal tissue sample, is categorized as being metastatic. The expression level of the polynucleotides may be determined by the expression level of mRNAs corresponding to the polynucleotides, or by the expression level of one or more polypeptides encoded by the polynucleotides. The tissue sample may be the primary gastric cancer tissue, metastatic gastric cancer tissue, or body fluid of a subject suspected of having gastric cancer.

In another embodiment, this invention provides antibodies that bind specifically to one or more polypeptides comprising amino acid sequences at least 85%, 86%, 87%, 88%, 89%, 90%, 91%, 92%, 93%, 94%, 95%, 96%, 97%, 98%, or 99% identical to amino acid sequences selected from SEQ ID NOs: 58-77 or a fragment thereof. A method of making the antibody comprises immunizing a non-human animal with the polypeptide. The antibodies can be used to detect, diagnose, and/or monitor metastatic gastric cancer, wherein the one or more antibodies are contacted with a tissue sample to detect the one or more polypeptides comprising amino acid sequences selected from SEQ ID NOs: 58-77 as described above.

In another embodiment, this invention provides a kit for use in detecting or diagnosing metastatic gastric cancer, comprising one or more antibodies that bind specifically to one or more polypeptides comprising amino acid sequences at least 85%, 86%, 87%, 88%, 89%, 90%, 91%, 92%, 93%, 94%, 95%, 96%, 97%, 98%, or 99% identical to amino acid sequences selected from SEQ ID NOs: 58-77 or a fragment thereof. Alternatively, the kit may comprise one or more probes comprising nucleotide sequences complementary to nucleotide sequences at least 90%, 91%, 92%, 93%, 94%, 95%, 96%, 97%, 98%, or 99% identical to nucleotide sequences selected from SEQ ID NOs: 37-57.

The invention thus provides a number of polynucleotides and polypeptides, which can be used as markers for detecting, diagnosing, and/or monitoring metastatic gastric cancer cells.

Additional objects and advantages of the invention will be set forth in part in the description which follows. The embodiments within the specification provide an illustration of embodiments of the invention and should not be construed to limit the scope of the invention. The accompanying drawings, which are incorporated in and constitute a part of, this specification illustrate several embodiments of the invention and, together with the description, serve to explain the principles of the invention.

### I. DEFINITIONS

The terms “detecting” and “diagnosing” are used interchangeably. By comparing the expression level of a metastatic gastric cancer gene in a tissue sample from a subject that has gastric cancer or is at risk of developing metastatic gastric cancer with the expression level of the same gene product in a normal tissue sample, one may determine, diagnose, or monitor whether there has been metastasis of the gastric cancer cells in the subject. If the expression level of the expressed gene product is higher or lower in the tissue sample from the subject than in the normal tissue sample, metastatic gastric cancer cells are “detected” in the subject or the subject is “diagnosed” with metastatic gastric cancer.

“Prognosis” means predicting the progression of disease based on the presence of increased and or decreased expression levels of the gene products of the disclosure. If an increase or decreased is detected, whether further treatment such as chemotherapy, radiotherapy, or surgery is required can be considered.

“Normal tissue samples” refers to gastric tissue and/or body fluid from a subject determined to be negative for gastric cancer, gastric cancer metastasis, or gastric tissue adjacent to gastric tumor from the same subject having the gastric tumor.

“Gene product” or “expression product” includes but is not limited to the mRNA corresponding to a gene and a polypeptide encoded by a gene.

“Monitoring” means periodically comparing the expression level of one or more metastatic gastric cancer genes in samples from a subject who has gastric cancer or is at the risk of developing metastatic gastric cancer with the expression levels of the genes from normal tissue, and determining whether metastasis is likely to occur and whether further treatment such as chemotherapy, radiotherapy, or surgery is required.

“Polynucleotide,” “nucleic acid,” “nucleic acid molecule,” “nucleic acid sequence,” “polynucleotide sequence,” “gene” and “nucleotide sequence” are used interchangeably to refer to polymeric forms of nucleotides of any length. The polynucleotides can comprise deoxyribonucleotides, ribonucleotides, and/or their analogs or derivatives. The term includes variants, which may include insertions, additions, deletions, or substitutions.

“Polypeptide,” “peptide,” and “protein,” are used interchangeably to refer to a polymeric form of amino acids of any length The term includes variants, which may include insertions, additions, deletions, or substitutions.

“Hybridizes specifically,” in the context of a polynucleotide, refers to hybridization under stringent conditions. Conditions that increase stringency of both DNA/DNA and DNA/RNA hybridization reactions are widely known and published in the art. Examples of stringent hybridization conditions include hybridization in 4× sodium chloride/sodium citrate (SSC), at about 65-70° C., or hybridization in 4×SSC plus 50% formamide at about 42-50° C., followed by one or more washes in 1×SSC, at about 65-70° C.

“Antibody” refers to an immunoglobulin molecule having a specific structure that interacts specifically with the antigen that was used to synthesize the antibody. The term “antibody” also refers to a fragment(s) of an antibody or a modified antibody, so long as it binds to one or more polypeptides encoded by the one or more polynucleotides described above. For instance, the antibody fragment may be Fab, F(ab′)2, Fv, or single chain Fv, in which Fv fragments from H and L chains are ligated by an appropriate linker. More specifically, an antibody fragment may be generated by treating an antibody with an enzyme, such as papain or pepsin. Alternatively, a gene encoding the antibody fragment may be constructed, inserted into an expression vector, and expressed in an appropriate host cell. The antibodies may include one or more radioactive, fluorescent, bioluminescent labels. The antibodies may also comprise an enzyme, such as horse radish peroxidase.

“Cancer” denotes any abnormal cell or tissue growth, for example, a tumor, whether malignant, pre-malignant, or non-malignant. It is characterized by uncontrolled proliferation of cells that may or may not invade the surrounding tissue and, hence, may or may not metastasize to new body sites. Cancer encompasses carcinomas, which are cancers of epithelial cells; carcinomas include squamous cell carcinomas, adenocarcinomas, melanomas, and hepatomas. Cancer also encompasses sarcomas, which are tumors of mesenchymal origin; sarcomas include osteogenic sarcomas, leukemias, and lymphomas. Cancers may involve one or more neoplastic cell type. The term cancer includes gastric cancer.

“Metastasis” means the migration of cancer cells from a primary tumor to sites elsewhere in the body. A tumor formed by cells that have spread is called a metastatic tumor or a metastasis.

“Purified” molecule refers to a molecule substantially free of cellular material or other contaminating molecules from the cell, tissue, or body fluid sources from which the molecule is derived.

“Isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

### II. Diagnoses and Prognoses

Detection of disease-specific biomarkers provides an effective screening strategy. Early detection provides not only early diagnosis, but in the case of cancer, can provide the ability to screen for polymorphisms and detect post-operative residual tumor cells and occult metastases, an early indicator of tumor recurrence. Early detection of disease-specific biomarkers indicative of metastasis can thus improve survival in patients before diagnosis, while undergoing treatment, and while in remission. Detection of the gene products of the disclosure can be used as a diagnostic or prognostic for diseases, including gastric cancer.

### III. Assay Formats

A. Detection of Polypeptides

The proteins produced by the genes of the disclosure can be detected using antibodies in a number of ways, including but not limited to (enzyme-linked immunosorbant assay) ELISA, Western blot, fluorescence, immunofluorescence, immunohistochemistry, or autoradiography. The antibodies used in such assays can be directly labeled or detected with a labeled secondary antibody. The antibodies may be used in a sandwich assay.

Labels include FITC, biotin, and radioisotopes, including, but not limited to ⁶⁴Cu, ⁶⁷Cu, ⁹⁰Y, ⁹⁹mTc, ¹¹¹In, ¹²⁴I, ¹²⁵I, ¹³¹I, ¹³⁷Cs, ¹⁸⁶Re, ²¹¹At, ²¹²Bi, ²¹³Bi, ²²³Ra, ²⁴¹Am, and ²⁴⁴Cm. Labels also include enzymes having detectable products (for example, luciferase, peroxidase, alkaline phosphatase, β-galactosidase, and the like). Labels further include fluorescers and fluorescent labels, fluorescence emitting metals, for example, ¹⁵²Eu, or others of the lanthanide series, electrochemiluniescent compounds, chemiluminescent compounds, for example, luminol, isoluminol, or acridinium salts, specific binding molecules, for example, magnetic particles, microspheres, and nanospheres. Such labels may be attached to proteins including antibodies, and may be attached to nucleic acids for use as probes or primers.

The method of preparing an antibody against a particular antigen is conventional and well known in the relevant art. In general, a laboratory animal is first immunized with a non-pasteurized preparation containing an antigenic polypeptide, treated with cyclophosphamide, and then injected with a pasteurized preparation again to boost the immune response. Following sacrifice of the lab animal, hybridomas are then generated and screened using known methods. Antibodies are then isolated by affinity chromatographic purification. Alternatively, an animal, such as a rabbit, is immunized with a composition comprising a particular antigen, and polyclonal antisera are obtained.

The antibody-based methods for detecting the proteins of the disclosure can be used in a format in which a single protein is detected with an antibody specific for that protein. Alternatively, multiple proteins can be detected simultaneously using a mixture of antibodies, wherein each antibody is specific for a different protein. In one embodiment, the multiple proteins are detected on a Western blot, and each protein identified by its apparent molecular weight. In another embodiment, each antibody is detected by a distinct means. For example, each antibody may comprise a fluorescent label with a unique emmission spectrum. In another embodiment, one protein might be detected by ELISA, while another protein could be detected by autoradiography. One or more antibodies to one or more genes products known not to change in metastasis, for example GAPDH, could be included in the mixture of antibodies as a control.

Antibodies that recognize the proteins produced by the genes of the disclosure may be specific, or may cross-react with related proteins. Accordingly, the methods of the disclosure include detecting proteins produced by variants of genes of the disclosure.

The proteins of the disclosure could also be detected using a protein array. For a review of protein array technology, see Kingsmore, “Multiplexed protein measurement: technologies and applications of protein and antibody arrays,” Nat Rev Drug Discov., 5: 310-321 (2006).

The proteins of the disclosure could be detected in cell extracts using mass-spectrometry. See e.g., Oshiro et al., “Parallel Identification of New Genes in Saccharomyces cerevisiae,” Genome Research, 12: 1210-20 (2002).

B. Detection of Nucleic Acids

The nucleic acids encoded by the genes of the disclosure can be detected through a variety of means including reverse transcription polymerase chain reaction (RT-PCR), real-time RT-PCR, multiplex PCR, TAQMAN® assay, Northern blotting, in situ hybridization, and microarray technology.

In Northern blotting formats, the means of detection include probes comprising isotopic and non-isotopic labels. One of skill in the art could combine multiple probes to simultaneously detect polynucleotides of the disclosure, wherein each probe comprises a distinct label. In one embodiment, each probe comprises a fluorescent label with a distinct emission spectrum. Alternatively, each probe has the same label and the polynucleotides are identified by electrophoretic mobility.

In methods using PCR, primers can be designed that are specific for the polynucleotides of the disclosure. Alternatively, primers could be designed that cross react with related polynucleotides. For example, stringent hybridization conditions could be chosen to allow hybridization to variants of the genes of the disclosure.

PCR-based formats could detect a single polynucleotide of the disclosure, or multiple polynucleotides simultaneously (“multi-plex” PCR). The amplification products of PCR can be detected by electrophoresis, including capillary electrophoresis, and staining with a dye, such as ethidium bromide. Each PCR product corresponding to genes of the invention could be identified by electrophoretic mobility. Alternatively, a label can be included in the PCR primer, and the PCR product detected based on the label. For example, a primer comprising a fluorescent label could be used, and the PCR product detected by detecting the label.

Where multiple polynucleotides are detected simultaneously, a mixture of primers each with a distinct label could used in a PCR reaction. The products produced by amplification of each polynucleotide are detected based on the label. For example, primers with labels with unique emission spectra could be used.

The polynucleotides of the disclosure could also be detected using microarray technology, as is well known in the art. Microarray assays have been widely used for rapid gene expression monitoring and sequence analysis at the genomic level. The term “microarray” refers to an ordered spatial arrangement of immobilized biomolecular probes arrayed on a solid supporting substrate. Typically, such arrays are oligonucleotide arrays comprising a nucleotide sequence that is complementary to at least one sequence that may be or is expected to be present in a biological sample. Such microarrays include spotted cDNA arrays, arrays comprising oligonucleotides, and arrays produced by photolithography such as those available from AFFYMETRIX®. For detailed descriptions about microarray technology, please refer to DNA Microarrays, Edited by M. Schena, In The Practical Approach Series, Series Editor: B. D. Hames (2000) Oxford University Press Inc., New York.

Polynucleotides produced by the genes of the disclosure could be detected by SAGE and by Massively Parallel Signature Sequencing (MPSS). See e.g., Brenner et al., “Gene expression analysis by massively parallel signature sequencing (MPSS) on microbead arrays,” Nature Biotechnology 18: 630-34 (2000).

### IV. Identification of Similar Sequences

The percentage of identity between a subject sequence and a reference standard can be determined by submitting both sequences to a computer analysis program with any parameters affecting the outcome of the alignment, set to the default position. In some instances, a subject sequence and the reference standard can exhibit the required percent identity without the introduction of gaps into one or both sequences. Furthermore, in many cases, polypeptides resulting from deletion, substitution, insertion and/or addition of one or more amino acids in the amino acid sequence of the desired protein would have the same functional activity as that of desired protein. Genes encoding such polypeptides are also included in the present disclosure and include both naturally occurring or artificial genes. In general, regarding the functional equivalents, there are many cases where genes encode products that are homologous to each other. Therefore, genes that can hybridize to the genes of the present disclosure and function in the same way are also included in the present disclosure.

Reference will now be made in detail to embodiments of the disclosure, examples of which are illustrated in the accompanying drawings.

### EXAMPLES

The following Examples are provided to illustrate certain aspects of the present disclosure and to aid those of skilled in the art in practicing this disclosure. These Examples are not to be considered to limit the scope of the disclosure.

### Statistical Analysis

Data were analyzed for significant differences using ANOVA followed by multiple comparisons with Student-Neuman-Keul Test. A difference between groups was considered to be significant when p<0.05.

### Example 1

### Selection of Highly Invasive MKN45 Sublines

A. EGFP Expressing MKN45 Cell Line

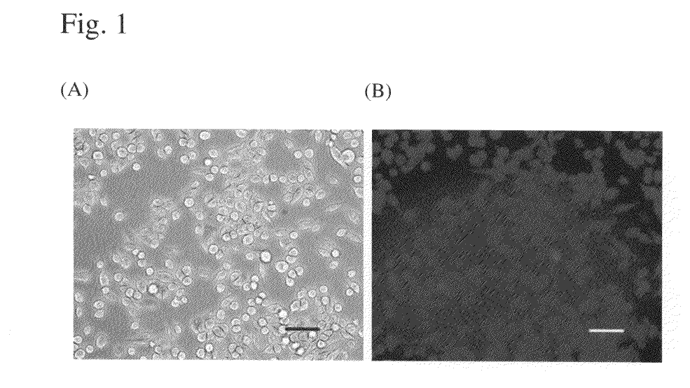

Human gastric cancer cell line MKN45 was transfected with a vector that encodes an enhanced green fluorescence protein (EGFP). The vector was delivered into MKN45 cells by LIPOFECTAMINE™ transfection and stable clones selected by serial dilution in G418 culture medium. The cells express EGFP driven by a cytomegalovirus (CMV) promoter and can be observed by green fluorescence in whole cells by microscopy. There was no difference in morphology of MKN45-GFP and MKN45 parental cells (FIG. 1).

B. Procedure for Producing MKN45 Cell Line

The human gastric cancer cell line MKN45 was acquired from the Japanese Collection of Research Bioresources/Human Science Research Resources Bank (Osaka, Japan). Based on a previous study, the cells were grown in RPMI 1640 with 10% fetal bovine serum (FBS) (Invitrogen, Carlsbad, Calif.) and 2 mM L-glutamine at 37° C., 5% CO₂. (Proc. Natl. Acad. Sci. 1994 91: 1858-62). Transfection was performed using LIPOFECTAMINE™ 2000 (Invitrogen, Carlsbad, Calif.). One day before transfection, MKN45 cells were seeded into 24-well culture plate and grown for 16-24 hr to 70%-90% confluence. For transfection, EGFP expression plasmid (pEGFP-C1, Cat. No. 6084-1) from Invitrogen was mixed with OPTI-MEM (Invitrogen, Carlsbad, Calif.) containing LIPOFECTAMINE™ 2000 at the ratio of pEGFP:OPTI-MEM:LIPOFECTAMINE™ 2000=2 μg:2 μl:100 μl. This mixture was then added directly to the cells, which were then returned to the CO₂ incubator at 37° C. After 48 hr of transfection, the cells were harvested and selected for stable GFP expression clone of MKN45 cells (MKN45-GFP) using both G418 from Invitrogen and a limiting dilution method.

C. Establishing Differential Metastatic Potential MKN45 Cell Lines

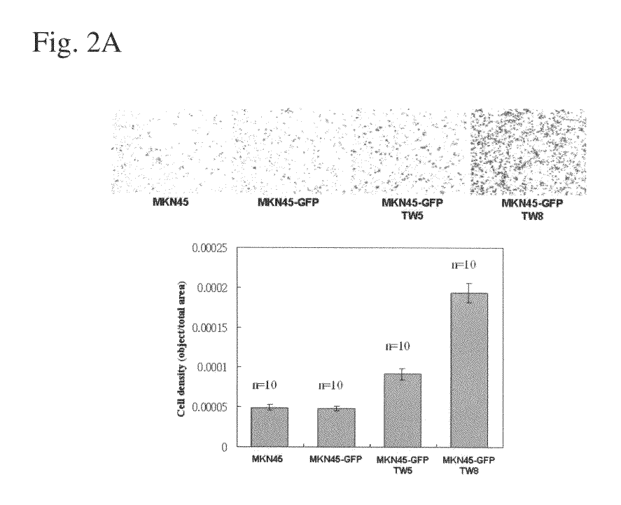

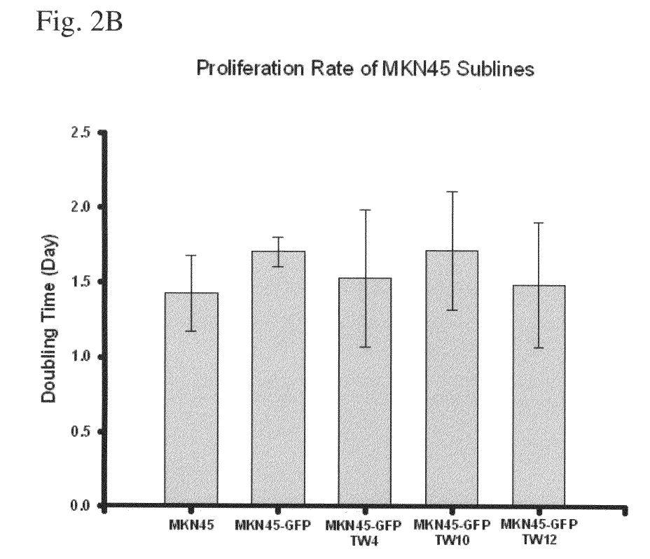

MKN45 cancer cell lines may become heterogeneous after long-term culture in vitro due to genetic instability. Thus, different characteristic MKN45 sublines may be isolated. To establish highly metastatic cell lines, MKN45-GFP cells were seeded into MATRIGEL™-coated TRANSWELL® chamber(s). After 72 hr incubation, the cells that had invaded MATRIGEL™ were collected and named as MKN45-GFP TW1, signifying one passage through the basal membrane matrix. Subsequently, these cells were amplified and repeatedly passed through the invasion-selection procedure up to 12 times. Cells could be passed through the invasion-selection procedure more than 12 times if desired. The cells from 4, 5, 8, 10, and 12 subsequent rounds of selection were harvested and designated MKN45-GFP TW4, TW5, TW8, TW10, and TW12, respectively. FIG. 2 shows the morphologic changes in cells under a microscope after selection. The MKN45 parental and MKN45-GFP cells had round morphology and less spindle shaped cells. Whereas the highly migrated TW sublines had more spindle shaped cells. In addition, the selected cells seemed less adhesive with other cells, and the parental cells were more aggregated than the selected cells.

D. Procedure for Selecting Invasive Cells with TRANSWELL® Plates

The GFP-expressing MKN45-GFP cells were selected for differential invasiveness using TRANSWELL® plates (Corning, Acton, Mass.). Briefly, the semi-permeable polycarbonate membranes (8.0 μm in pore size) of the 24-well inserts of TRANSWELL® were coated with 50 μl of reconstituted basement-membrane matrix (MATRIGEL™, 1:1 dilution with complete medium) from BD Biosciences. MKN45-GFP cells were resuspended in RPMI 1640 containing 10% FBS and seeded onto the upper-surface of the MATRIGEL™-coated membrane. Following incubation for 72 hr at 37° C., the inserts were removed. The cells that migrated and invaded through the polycarbonate membranes and attached to the surface of well bottom were harvested aseptically and grown for further selection processes. The arrested invasive MKN45-GFP subline after the first-round selection was named as MKN45-GFP TW1, and thus sublines from 4th, 10th, and 12th rounds of the same selection process were named as MKN45-GFP TW4, MKN45-GFP TW10, and MKN45-GFP TW12, respectively.

E. In Vitro Invasion Assay and Cell Growth of MKN45 Sublines

Measurement of the difference in invasiveness associated with MKN45 parental, MKN45-GFP, MKN45-GFP TW5, and MKN45-GFP TW8 was performed with a MATRIGEL™ coated 6-well TRANSWELL®. The invasive potential was determined on the basis of cells' ability to invade a matrix barrier containing major components of the basement membrane. The results obtained from 10 repetitions with each subline showed that the invasive potential had increased by 2-fold for invasion-selected MKN45-GFP TW5 and 4-fold for MKN45-GFP TW8 sublines as compared with the MKN45-GFP cells. There was no difference between MKN45-GFP and its parental cells (FIG. 2A lower panel). FIG. 2A upper panel shows the results of an invasion assay in which the lower sides of the TRANSWELL® filters were stained with hematoxylin and observed by microscopy. There were more cells that invaded the lower side of the membrane in MKN45-GFP TW5 and TW8 sublines. Cell proliferation analysis revealed that highly invasive subline MKN45-GFP TW10 did not show any significant increase in cell growth (FIG. 2B).

F. Procedure for In Vitro Invasion Assay

The concept of using basement membrane coated TRANSWELL® to quantify tumor cell invasion is well established. (Proc. Natl. Acad. Sci. 1994 91: 1858-62). Cell invasion was evaluated using 1:30 diluted MATRIGEL™ (BD Biosciences, San Jose, Calif.) coated 6-well TRANSWELL® plates. Cells were suspended into the upper-chamber at a concentration of 1×0⁶/ml in 1 ml of RPMI 1640 with 10% FBS. The lower-chamber contained 2 ml of RPMI 1640 supplemented with 10% FBS. After incubation for 24 hr at 37° C., cells that invaded through the coated MATRIGEL™ and membrane to the lower surface of the membrane and the button surface of the culture well were fixed with 3.7% formaldehyde in phosphate-buffered saline (PBS). Cells and MATRIGEL™ on the upper side of the membrane were scraped with a cotton swab. After fixation, the cells were stained with hematoxylin and washed by PBS. The cell number in the lower side of the membrane was obtained using images captured and analyzed with Image Pro Plus (Media Cybernetics, Silver Spring, Md.).

G. Procedure for Cell Growth Assay

Use of a tetrazolium salt (MTS)/phenazine methosulfate (PMS) assay to evaluate cell growth rate was well established in our previous study (J. Med. Chem. 2003, 46, 1706-15), MKN45, MKN45-GFP, MKN45-GFP TW4, MKN45-GFP TW10, and MKN45-GFP TW12 cells were seeded at 2000, 4000, and 6000 cells in 96-well plates (Corning, Acton, Mass.) in triplicate. The cells were harvested and monitored every 24 hr for 6 days by MTS/PMS assay (2 mg/ml MTS (Promega, Madison, Wis.) and 0.38 mg/ml PMS (Sigma, Saint Louis, Mo.) in phenol red free RPMI1640 medium, detected OD at 490 nm after 90 min 37° C. incubation). The in vitro cell doubling time was obtained by a nonlinear regression (equation: f=a*2̂(x/b)) using SigmaPlot 2001 (Systat Software, San Jose, Calif.).

### Example 2

### Identification of Gastric Cancer Metastasis-Related Genes By RNA Microarray

A. RNA Microarray Assay

Total RNAs were isolated from MKN45 and its sublines using TRIZOL® reagent (Invitrogen, Carlsbad, Calif.) following the manufacturer's protocol. The purified RNA was quantified in optical density at 260 nm by a ND-1000 spectrophotometer from Nanodrop Technology (Wilmington, Del.) and qualitated by Bioanalyzer 2100 from Agilent Technology (Santa Clara, Calif.). 0.5 μg of total RNA was amplified by a low RNA input fluor linear amp kit (Agilent Technologies, Santa Clara, Calif.) and labeled with Cy3 or Cy5 (CyDye; Perkin Elmer, Waltham, Mass.) during in vitro transcription. The sample RNA was labeled by Cy5 and RNA from Universal Human Reference RNA was labeled by Cy3. 2 g of Cy-labled cRNA was fragmented to an average size of about 50-100 nucleotides by incubation with fragmentation buffer at 60° C. for 30 min. Correspondingly fragmented labeled cRNA was then pooled and hybridized to Human 1A (version 2) oligo microarray (Agilent Technologies, Santa Clara, Calif.) at 60° C. for 17 hr. After washing and drying by nitrogen gun blowing, microarrays were scanned with Agilent microarray scanner at 535 nm for Cy3 and 625 nm for Cy5, respectively. Scanned images were analyzed by an image analysis and normalization software Feature extraction 8.1 from Agilent Technologies to quantify signal and background intensity, substantially normalized the data by rank-consistency-filtering LOWESS method.

Total RNA extracted from MKN45 parental, MKN45-GFP and its selected sublines TW4, TW10, and TW12 were quantified and qualitated by spectrophotometer and Bioanalyzer 2100 respectively. Microarray were performed by hybridizing with Human 1A oligo include 20 thousand putative genes to profile the gene expression patterns and using Cy3 and Cy5 signal to compare experimental group (MKN45 parental, MKN45-GFP TW4, TW10, and TW12) expression level with control (MKN45-GFP).

B. Clustering of Microarray Data

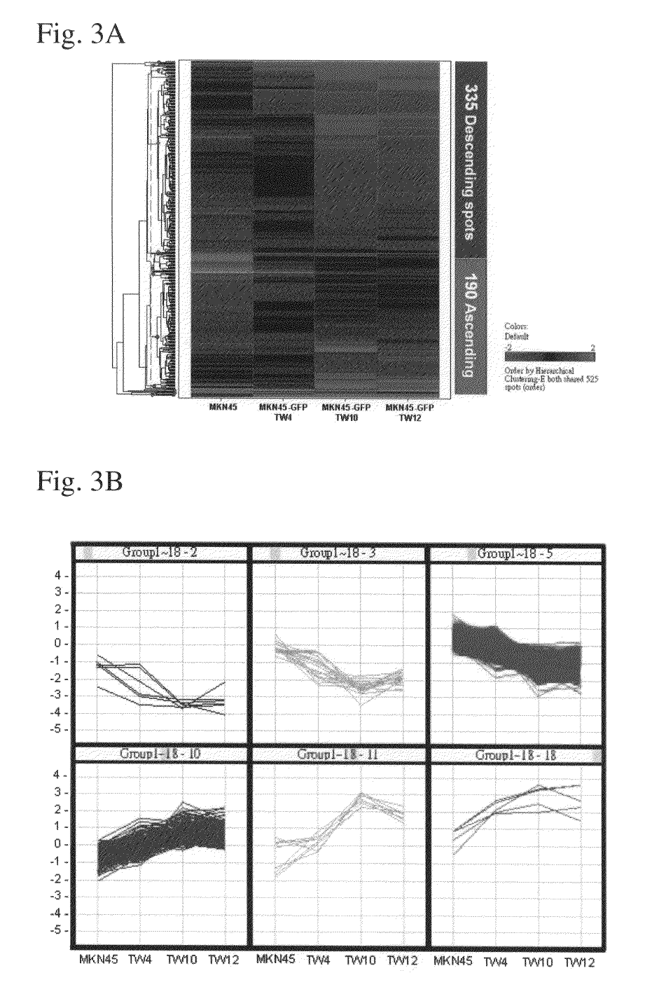

The expression levels of the genes represented on the microarray were correlated (p<0.05) with the invasive abilities of cell lines. FIG. 3A shows a hierarchical clustering analysis image with 525 spots (including redundant spots) from significant expression values which share similar tendency containing 190 ascending spots (positive correlation with invasiveness) and 335 descending spots (negative correlation with invasiveness). The expression levels were pseudocolor encoded. The upper part of image shows the levels of descending genes expression from high (red) to low (green), and the lower part shows the levels of ascending genes in the opposite direction. Based on hierarchical clustering calculation, the pattern of 525 spots can be divided into 18 groups. The genes in a group share similar expression profiles and predicted cellular and subcellular regulatory mechanisms, suggesting similar functional phenotypes associated with gastric cancer biology and/or tumor metastasis. The part of FIG. 3A shows three groups of expression profiles that correlated positively with the invasiveness of the cell lines. Each of the three groups included expression profiles of 163, 8, and 5 spots, respectively. The lower part of FIG. 3A shows three groups of expression profiles which had negative correlation with invasiveness, and each groups included 3, 15, and 273 spots, respectively. FIG. 3B shows three groups of ascending and descending expression profiles from eighteen groups.

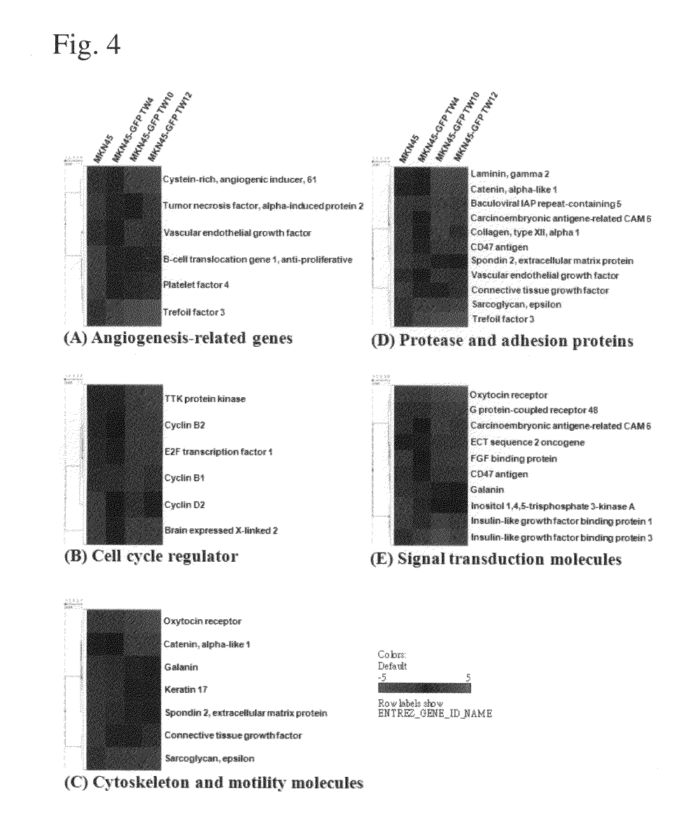

The genes clustered in FIG. 3 were classified into five categories on the basis of their cellular functions associated with tumor progression (FIG. 4). These categories included angiogenesis-related genes such as angiogenic inducer Cyr61 and vesicular endothelial growth factor, cell cycle regulators such as TTK protein kinase and cyclin B2; cytoskeleton and motility molecules such as oxytocin receptor and catenin alpha-like 1; protease and adhesion molecules such as laminin gamma 2 and collagen type XII alpha 1, and signal transduction molecules such as G protein-coupled receptor 48 and heparin-binding growth factor binding protein. Genes with multiple roles were included in more than one category.

C. Identification of Gastric Cancer Metastasis-Related Genes

76 genes related to gastric cancer metastasis in the selected MKN45 invasive sublines were identified using the above microarray analysis, with at least 3-fold differences in their mean expression levels from that of normal cells. Among these 76 identified genes, 22 genes have never been disclosed in the literature pertaining to gastric cancer or metastasis. The microarray probes (SEQ ID NO: 15-36) for identifying these novel genes are provided in Table 1, as well as the coding sequences (“CDS”; SEQ ID NOs: 37-57), amino acid sequences (SEQ ID NOs: 58-74), and complete cDNA sequences of the genes.

TABLE 1
The probe sequences for identifying novel gastric cancer
metastasis-related genes

| | | Probe | | Amino Acid |
|---|---|---|---|---|
| | | Sequence | CDS | Sequence |
| Gene | Title | (SEQ ID NO:) | (SEQ ID NO:) | (SEQ ID NO:) |
| RIC3 | RIC3 protein | 15 | 37 | 58 |
| CHCHD5 | coiled-coil-helix-coiled-coil-helix | 16 | 38 | 59 |
| | domain containing 5 | | | |
| SAMD9 | FLJ20073 protein | 17 | 39 | 60 |
| CXorf26 | hypothetical protein MGC874 | 18 | 40 | 61 |
| SLC22A17 | solute carrier family 22 (organic | 19 | 41 | 62 |
| | cation transporter), member 17 | | | |
| ZNF572 | zinc finger protein 572 | 20 | 42 | 63 |
| THC2052903 | THC2052903 | 21 | 43 | N/A |
| ATAD2 | ATPase family, AAA domain | 22 | 44 | 64 |
| | containing 2 | | | |
| RIBC2 | chromosome 22 open reading | 23 | 45 | 65 |
| | frame 11 | | | |
| NEIL3 | DNA glycosylase hFPG2 | 24 | 46 | 66 |
| HECTD2 | HECT domain containing 2 | 25 | 47 | 67 |
| FLJ32065 | hypothetical protein FLJ32065 | 26 | 48 | 68 |
| C14orf151 | hypothetical protein MGC13251 | 27 | 49 | 69 |
| KIF18A | kinesin family member 18A | 28 | 50 | 70 |
| PGM2L1 | phosphoglucomutase 2-like 1 | 29 | 51 | 71 |
| TSPAN1 | tetraspan 1 | 30 | 52 | 72 |
| WDHD1 | WD repeat and HMG-box DNA | 31 | 53 | 73 |
| | binding protein 1 | | | |
| SEC11L3 | similar to signal peptidase complex | 32 | 54 | 74 |
| | (18 kD) | | | |
| THC2095000 | THC2095000 | 33 | N/A | N/A |
| A_23_P111766 | A_23_P111766 | 34 | 55 | 75 |
| BEX2 | brain expressed X-linked 2 | 35 | 56 | 76 |
| KBTBD9 | kelch repeat and BTB (POZ) | 36 | 57 | 77 |
| | domain containing 9 | | | |

TABLE 2
The identified gastric cancer metastasis-related genes and the
encoded proteins

| | | Unigene | Systematic |
|---|---|---|---|
| Gene | Title | Cluster | Name |
| RIC3 | RIC3 protein | Hs.458375 | AY326436 |
| CHCHD5 | coiled-coil-helix-coiled-coil-helix | Hs.375707 | NM_032309 |
| | domain containing 5 | | |
| SAMD9 | FLJ20073 protein | Hs.65641 | NM_017654 |
| CXorf26 | hypothetical protein MGC874 | Hs.370100 | NM_016500 |
| SLC22A17 | solute carrier family 22 (organic | Hs.373498 | NM_016609 |
| | cation transporter), member 17 | | |
| ZNF572 | zinc finger protein 572 | Hs.175350 | NM_152412 |
| THC2052903 | THC2052903 | Unknown | THC2052903 |
| ATAD2 | ATPase family, AAA domain | Hs.298646 | NM_014109 |
| | containing 2 | | |
| RIBC2 | chromosome 22 open reading | Hs.144505 | NM_015653 |
| | frame 11 | | |
| NEIL3 | DNA glycosylase hFPG2 | Hs.405467 | NM_018248 |
| HECTD2 | HECT domain containing 2 | Hs.437398 | NM_173497 |
| FLJ32065 | hypothetical protein FLJ32065 | Hs.396447 | NM_153032 |
| C14orf151 | hypothetical protein MGC13251 | Hs.317821 | NM_032714 |
| KIF18A | kinesin family member 18A | Hs.301052 | NM_031217 |
| PGM2L1 | phosphoglucomutase 2-like 1 | Hs.26612 | NM_173582 |
| TSPAN1 | tetraspan 1 | Hs.38972 | NM_005727 |
| WDHD1 | WD repeat and HMG-box DNA | Hs.385998 | NM_007086 |
| | binding protein 1 | | |
| SEC11L3 | similar to signal peptidase | Hs.45107 | NM_033280 |
| | complex (18 kD) | | |
| THC2095000 | THC2095000 | Unknown | THC2095000 |
| A_23_P111766 | A_23_P111766 | Unknown | A_23_P111766 |
| BEX2 | brain expressed X-linked 2 | Hs.398989 | NM_032621 |
| KBTBD9 | kelch repeat and BTB (POZ) | Hs.348392 | AB067508 |
| | domain containing 9 | | |

### Example 3

### Confirmation of Microarray Result Using RT-PCR and Western Blot

A. Real Time Reverse Transcription PCR (RT-PCR) Analysis

Briefly, samples of the total RNA (1-5 μg) from MKN45 and its selected sublines were reverse-transcribed in a total volume of 20 μl by the SuperScript III First-Strand Synthesis System (Cat. No. 18080-400, Invitrogen, Carlsbad, Calif.). The reverse transcription products (1 μg) were used directly for PCR amplification. PCR amplification was performed with the PCR Reagent System (Cat. No. 10198-018, Invitrogen) in accordance with the manufacturer's instructions in a PC818 Program Temp. Control System is from ASTEC (Fukuoka, Japan). Oligonucleotide cDNA primers used for amplification of the selected genes are listed in Table 3.

TABLE 3

The Sequences of cDNA primers for the selected
genes

| | | |
|---|---|---|
| | | SEQ |
| | | ID |
| Name | Sequence | NO: |

| | | |
|---|---|---|
| OTR | 5′-CCTTCATCGTGTGCTGGACG-3′ (forward) | 1 |
| | 5′-CTAGGAGCAGAGCACTTATG-3′ (reverse) | 2 |
| LGR4 | 5′-GGGAAGCTGGATGATTCGTCTTACT-3′ (forward) | 3 |
| | 5′-GAAAAGGGGAAAACAGCCTGCT-3′ (reverse) | 4 |
| TFF3 | 5′-AGAGCCTTCCCCAAGCAAACA-3′ (forward) | 5 |
| | 5′-GCAGGGGCTTGAAACACCAA-3′ (reverse) | 6 |
| BEX2 | 5′-CCTTGGCCCTACCTTTGAATGT-3′ (forward) | 7 |
| | 5′-TGCTGACTGCCCGCAAACTA-3′ (reverse) | 8 |
| SGCE | 5′-TTCTCCAAGGTACACTCCGATCG-3′ (forward) | 9 |
| | 5′-GGCCGATGTGATGTTTATGGC-3′ (reverse) | 10 |
| IGFBP3 | 5′-ACGAGTCTCAGAGCACAGATACCC-3′ (forward) | 11 |
| | 5′-TATCCACACACCAGCAGAAGCC-3′ (reverse) | 12 |
| GAPDH | 5′-TCCACCACCCTGTTGCTGTA-3′ (forward) | 13 |
| | 5′-ACCACAGTCCATGCCATCAC-3′ (reverse) | 14 |

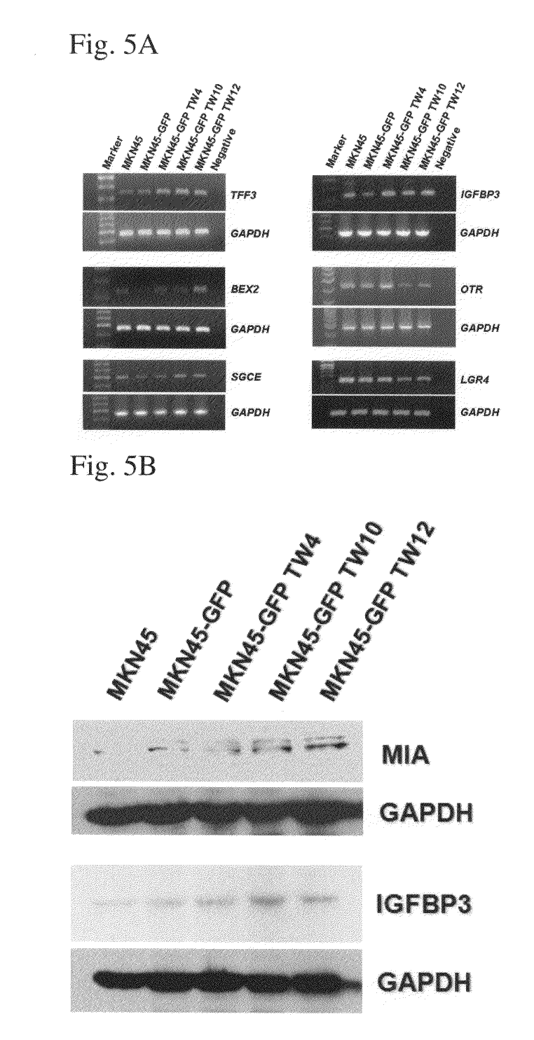

FIG. 5A illustrates the RNA expression level of the six selected genes, in which TFF3, BEX2, SGCE, and IGFBP3 genes had higher expression levels in the more invasive cell line, i.e., MKN45-GFP TW12. On the other hand, the OTR and LGR4 genes were highly expressed in the less invasive cell line, i.e., MKN45-GFP. These results of RT-PCR analysis were consistent with those from the microarray studies.

B. Western Blot of Protein Expression in Clustered Genes

To demonstrate that the protein expression of identified genes was also consistent with microarray analysis, two antibodies (melanoma-inhibitory activity and insulin-like growth factor binding protein 3) were used to perform Western blot analysis for all five MKN45 sublines. Each experiment was carried out in triplicate. FIG. 5B shows the protein expression level of melanoma-inhibitory activity (MIA) and insulin-like growth factor binding protein 3 (IGFBP3) of five MKN45 sublines, respectively. The protein expression level of MIA was higher in highly invasiveness MKN45 subline (MKN45-GFP TW12) in both of pro-form and mature-form. The IGFBP3 gene which has positive correlated RNA expression level with invasive ability of MKN45 sublines was also up-regulated in protein level. These results demonstrate that the Western blot analysis of protein were consistent with microarray analysis.

C. Procedure for Western Blot Analysis

MKN45 and its selected sublines (MKN45-GFP, MKN45-GFP TW4, MKN45-GFP TW10, and MKN45-GFP TW12) were cultured and the cells were lysed by mixing with RIPA (Radio-Immunoprecipitation Assay, Cat. No. R0278, Sigma) buffer containing Proteinase Inhibitor Cocktail (Cat. No. P8340, Sigma). The protein amounts in the cell lysates were quantified by a bicinchoninic acid (BCA) protein assay kit (PIERCE, Rockford, Ill.) and mixed with electrophoresis loading buffer (50 mM Tris-HCl, 2% dodecylsulfate sodium salt (SDS), 0.1% bromophenol blue, 10% glycerol, and 1 mM dithiothreitol (DTT)). The samples were electrophoresed on 12% SDS-polyacrylamide gel under reducing conditions. After electrophoresis, the proteins were transferred electrophoretically to PVDF membrane (IMMOBILON®-P) from Millipore. The membrane was blocked with 5% skim milk for 30 min at 37° C. After the blocking process, the membrane was incubated overnight with mouse monoclonal antibodies against human melanoma-inhibitory activity (MIA) or human insulin-like growth factor binding protein three (IGFBP3) at 4° C. Both antibodies were purchased from R&D System (Minneapolis, Minn.). The membrane was washed three times with PBS-T buffer for five min each, and then incubated with HRP-conjugated donkey anti-mouse IgG for one hr at room temperature. After washed with PBS-T, the membrane was incubated with the WESTERN LIGHTNING® ECL detecting reagent from Perkin Elmer (Waltham, Mass.).

FIG. 5B illustrates the protein expression level of melanoma-inhibitory activity (MIA) and insulin-like growth factor binding protein 3 (IGFBP3) of the MKN45 and its invasive sublines. The protein expression level of MIA was higher in highly invasive MKN45 subline (i.e., MKN45-GFP TW12) in its pro-form and its mature-form. The IGFBP3 gene, which had positively correlated RNA expression level with invasive ability of MKN45 sublines was also up-regulated in protein level. These results demonstrated that the Western blot analysis of protein were consistent with microarray analysis.

### Example 4

### Detection of Gastric Cancer Metastasis in Patients

A. Nucleic Acid-based Methods of Detection

A sample of primary gastric cancer tissue and a sample of non-gastric tissue (normal) are taken from a patient suffering from gastric cancer. Total RNA is isolated from the samples using TRIZOL® reagent (Invitrogen, Carlsbad, Calif.) following the manufacturer's protocol. The purified RNA is quantified in optical density at 260 nm by a ND-1000 spectrophotometer from Nanodrop Technology (Wilmington, Del.) and qualitated by Bioanalyzer 2100 from Agilent Technology (Santa Clara, Calif.). 0.5 μg of total RNA from the cancer sample is amplified by a low RNA input fluor linear amp kit (Agilent Technologies, Santa Clara, Calif.) and labeled with Cy3 (CyDye; Perkin Elmer, Waltham, Mass.) during in vitro transcription. The RNA from the normal sample is labeled with Cy5. 2 g of Cy-labled cRNA is fragmented to an average size of about 50-100 nucleotides by incubation with fragmentation buffer at 60° C. for 30 min. The fragmented labeled cRNA is then pooled and hybridized to Human 1A (version 2) oligo microarray (Agilent Technologies, Santa Clara, Calif.) at 60° C. for 17 hr. After washing and drying by nitrogen gun blowing, microarrays are scanned with an Agilent microarray scanner at 535 nm for Cy3 and 625 nm for Cy5, respectively. Scanned images are then analyzed by an image analysis and normalization software Feature extraction 8.1 from Agilent Technologies to quantify signal and background intensity, substantially normalized the data by rank-consistency-filtering LOWESS method. The above method is repeated in which the Cy3 and Cy5 labels are reversed.

The relative expression levels of the polynucleotides reveals that one or more polynucleotides comprising the sequence of SEQ ID NOs: 37-53 are expressed at least three-fold lower in the cancer sample than the normal sample, and/or one or more polynucleotides comprising the sequence of SEQ ID NOs: 54-57 are expressed at least three-fold higher in the cancer sample than in the normal sample.

B. Antibody-Based Methods of Detection

A sample of primary gastric cancer tissue and a sample of non-gastric tissue (normal) are taken from a patient suffering from gastric cancer. Proteins are isolated from each sample and added to individual wells of a microtiter plate. Total proteins can be extracted form blood, tissue, urine or other body fluid using commercially available kits (e.g. Calbiochem PROTEOEXTRACT™ Complete Mammalian Proteome Extraction Kit) or RIPA buffer extraction. To each well is added a primary antibody that binds to a polypeptide of the disclosure. The microtiter plate is incubated to allow binding of antibody with polypeptide The titers and incubation conditions are varied for each antibody, depending on antibody-polypeptide affinity, antibody specificity and sensitivity as is well known in the art. For example, the primary antibody is hybridized for 1 hr at room temperature or O/N at 4° C. A secondary antibody which binds to the primary antibodies is added to each well. The secondary antibody comprises Horse Radish Peroxidase (HRP). The mixture comprising the proteins, primary and secondary antibodies is incubated to allow binding between the primary and secondary antibodies. For example, the secondary antibody was hybridized for 1 hr at room temperature. An ECL HRP substrate, is added to each well. The microtiter plate is inserted into a plate reader and the product of the HRP reaction is measured to determine the level of polypeptide in each well.

The relative expression levels of the polypeptides reveals that one or more polypeptides comprising the sequence of SEQ ID NOs: 58-73 are expressed at least three-fold lower in the cancer sample than the normal sample, and/or one or more polypeptides comprising the sequence of SEQ ID NOs: 74-77 are expressed at least three-fold higher in the cancer sample than in the normal sample.

The foregoing description of embodiments of the disclosure is presented for the purpose of illustration and description. It is not intended to be exhaustive or to limit the disclosure to the precise embodiments disclosed. Numerous modifications or variations are possible in light of the above teachings. The embodiments discussed were chosen and described to illustrate the principles of the disclosure and its practical application to thereby enable one of ordinary skill in the art to utilize the disclosure in various embodiments and with various modifications as are suited to the particular use contemplated. All such modifications and variations are within the scope of the disclosure as determined by the appended claims.

What is claimed is:

1. A method of detecting metastatic gastric cancer comprising:
a) obtaining one or more tissue samples from a subject;
b) measuring in the tissue samples the levels of one or more polynucleotides comprising nucleotide sequences at least 90% identical to nucleotide sequences chosen from SEQ ID NOs: 37-53;
c) determining whether the expression levels of the one or more polynucleotides are lower than the expression levels of the one or more polynucleotides in normal tissue samples, wherein a lower expression level indicates the presence of metastatic gastric cancer.

2. The method of claim 1, wherein the one or more polynucleotides comprise nucleotide sequences chosen from SEQ ID NOs: 37-53.

3. The method of claim 1, wherein the expression levels of the one or more polynucleotides are determined by a method chosen from:
a) hybridizing one or more probes to the one or more polynucleotides and measuring the amount of the one or more probes bound to the one or more polynucleotides;
b) amplifying the polynucleotides using PCR and measuring the levels of the PCR products;
c) Serial Analysis of Gene Expression (SAGE); and
d) Massively Parallel Signature Sequencing (MPSS).

4. The method of claim 1, wherein an expression level of the one or more polynucleotides in the sample is at least three-fold lower than a level of the one or more polynucleotides in normal tissue samples.

5. The method of claim 1, wherein the tissue sample is chosen from primary gastric cancer tissue, metastatic gastric cancer tissue, and body fluid of the subject.

6. The method of claim 5, wherein the body fluid is chosen from blood, plasma, serum, peritoneal fluid, urine, and saliva.

7. A method of detecting metastatic gastric cancer comprising:
a) obtaining one or more tissue samples from a subject;
b) measuring in the tissue samples the expression levels of one or more polypeptides comprising an amino acid sequence at least 90% identical to amino acid sequences chosen from SEQ ID NOs: 58-73;
c) determining whether the expression levels of the one or more polypeptides in the are lower than the expression levels of the one or more polypeptides in normal tissue samples, wherein a lower expression level indicates the presence of metastatic gastric cancer cells.

8. The method of claim 7, wherein the one or more polypeptides comprise amino acid sequences chosen from SEQ ID NOs: 58-73.

9. The method of claim 7, wherein the levels of the one or more polypeptides are determined by a method chosen from:
a) contacting the one or more polypeptides with one or more antibodies and detecting one or more complexes comprising the one or more polypeptides and the one or more antibodies;
b) mass-spectrometry;
c) hybridization to a protein array.

10. The method of claim 7, wherein the tissue sample is chosen from primary gastric cancer tissue, metastatic gastric cancer tissue, and body fluid of the subject.

11. The method of claim 7, wherein the body fluid is chosen from blood, plasma, serum, peritoneal fluid, urine, and saliva.

12. A method of detecting metastatic gastric cancer comprising:
a) obtaining one or more tissue samples from a subject;
b) measuring in the tissue samples the expression levels of one or more polynucleotides, wherein the one or more polynucleotides comprise nucleotide sequences at least 90% identical to nucleotide sequences chosen from SEQ ID NOs: 54-57;
c) determining whether the expression levels of the one or more polynucleotides in the tissue samples is higher than the expression levels of the one or more polynucleotides in normal tissue samples, wherein a higher expression level indicates the presence of metastatic gastric cancer cells.

13. The method of claim 12, wherein the one or more polynucleotides comprise nucleotide sequences chosen from SEQ ID NOs: 54-57.

14. The method of claim 12, wherein the expression levels of the one or more polynucleotides are determined by a method chosen from:
a) hybridizing one or more probes to the one or more polynucleotides and measuring the amount of the one or more probes bound to the one or more polynucleotides;
b) amplifying the polynucleotides using PCR and measuring the levels of the PCR products;
c) Serial Analysis of Gene Expression (SAGE); and
d) Massively Parallel Signature Sequencing (MPSS).

15. The method of claim 12, wherein an expression level of the one or more polynucleotides in the sample is at least three-fold higher than a level of the one or more polynucleotides in normal tissue samples.

16. A method of detecting metastatic gastric cancer comprising:
a) obtaining one or more tissue samples from a subject;
b) measuring in the tissue samples the expression levels of one or more polypeptides, comprising polypeptide sequences at least 90% identical to polypeptide sequences chosen from SEQ ID NOs: 74-77;
c) determining whether the expression levels of the one or more polypeptides in the tissue samples are higher than the expression levels of the one or more polypeptides in normal tissue samples, wherein a higher expression level indicates the presence of metastatic gastric cancer cells.

17. The method of claim 16, wherein the one or more polypeptides comprise amino acid sequences chosen from SEQ ID NOs: 74-77.

18. The method of claim 16, wherein the levels of the one or more polypeptides are determined by a method chosen from:
a) contacting the one or more polypeptides with one or more antibodies and detecting one or more complexes comprising the one or more polypeptides and the one or more antibodies;
b) mass-spectrometry;
c) hybridization to a protein array.

19. The method of claim 16, wherein the tissue sample is chosen from primary gastric cancer tissue, metastatic gastric cancer tissue, and body fluid of the subject.

20. The method of claim 16, wherein the body fluid is chosen from blood, plasma, serum, peritoneal fluid, urine, and saliva.

21. A kit comprising a composition chosen from:
a) one or more polynucleotides complementary to nucleotide sequences at least 90% identical to nucleotide sequences comprising SEQ ID NOs: 37-57; and
b) one more antibodies specific for a polypeptide chosen from polypeptides at least 90% identical to polypeptide sequences comprising SEQ ID NOs: 58-77.

22. A tumor cell line chosen from MKN45-GFP TW4, as deposited with ______ under the accession number ______ on ______, MKN45-GFP TW5, as deposited with ______ under the accession number ______ on ______, MKN45-GFP TW8, as deposited with ______ under the accession number ______ on ______, MKN45-GFP TW10, as deposited with ______ under the accession number ______ on ______, and MKN45-GFP TW12, as deposited with ______ under the accession number ______ on ______.


SEQUENCE LISTING (77 sequences)

SEQ ID NO: 1 - DNA, 20 bp, Artificial Sequence
  Description of Artificial Sequence Synthetic primer
ccttcatcgtgtgctggacg

SEQ ID NO: 2 - DNA, 20 bp, Artificial Sequence
  Description of Artificial Sequence Synthetic primer
ctaggagcagagcacttatg

SEQ ID NO: 3 - DNA, 25 bp, Artificial Sequence
  Description of Artificial Sequence Synthetic primer
gggaagctggatgattcgtcttact

SEQ ID NO: 4 - DNA, 22 bp, Artificial Sequence
  Description of Artificial Sequence Synthetic primer
gaaaaggggaaaacagcctgct

SEQ ID NO: 5 - DNA, 21 bp, Artificial Sequence
  Description of Artificial Sequence Synthetic primer
agagccttccccaagcaaaca

SEQ ID NO: 6 - DNA, 20 bp, Artificial Sequence
  Description of Artificial Sequence Synthetic primer
gcaggggcttgaaacaccaa

SEQ ID NO: 7 - DNA, 22 bp, Artificial Sequence
  Description of Artificial Sequence Synthetic primer
ccttggccctacctttgaatgt

SEQ ID NO: 8 - DNA, 20 bp, Artificial Sequence
  Description of Artificial Sequence Synthetic primer
tgctgactgcccgcaaacta

SEQ ID NO: 9 - DNA, 23 bp, Artificial Sequence
  Description of Artificial Sequence Synthetic primer
ttctccaaggtacactccgatcg

SEQ ID NO: 10 - DNA, 21 bp, Artificial Sequence
  Description of Artificial Sequence Synthetic primer
ggccgatgtgatgtttatggc

SEQ ID NO: 11 - DNA, 24 bp, Artificial Sequence
  Description of Artificial Sequence Synthetic primer
acgagtctcagagcacagataccc

SEQ ID NO: 12 - DNA, 22 bp, Artificial Sequence
  Description of Artificial Sequence Synthetic primer
tatccacacaccagcagaagcc

SEQ ID NO: 13 - DNA, 20 bp, Artificial Sequence
  Description of Artificial Sequence Synthetic primer
tccaccaccctgttgctgta

SEQ ID NO: 14 - DNA, 20 bp, Artificial Sequence
  Description of Artificial Sequence Synthetic primer
accacagtccatgccatcac

SEQ ID NO: 15 - DNA, 60 bp, Artificial Sequence
  Description of Artificial Sequence Synthetic probe
agccatgatttcagtttcacataagaatgtttactcaatgtttaagtgtgttgccccaaa

SEQ ID NO: 16 - DNA, 60 bp, Artificial Sequence
  Description of Artificial Sequence Synthetic probe
agatggagtcctgagccctggacatgggcccggctttcctggatatcaggacttccaata

SEQ ID NO: 17 - DNA, 60 bp, Artificial Sequence
  Description of Artificial Sequence Synthetic probe
tcactggaggaagattttcccttgcttctgcataaaattttaactccataacttataagc

SEQ ID NO: 18 - DNA, 60 bp, Artificial Sequence
  Description of Artificial Sequence Synthetic probe
ttactctgagttccggaaaaattttgagacccttaggatagatgtgttggacccagaaga

SEQ ID NO: 19 - DNA, 60 bp, Artificial Sequence
  Description of Artificial Sequence Synthetic probe
cttgctctatcattctgtttcaataaagacatttggaataaacgagcatatcatagcctg

SEQ ID NO: 20 - DNA, 60 bp, Artificial Sequence
  Description of Artificial Sequence Synthetic probe
ttgcatgggggttattttatctttcatgattgtggtgcacctgatgctggcggggtattt

SEQ ID NO: 21 - DNA, 60 bp, Artificial Sequence
  Description of Artificial Sequence Synthetic probe
atgtcatttgccatattgccatctaaaggagaaaaactgcatcagcaaagccattgtatt

SEQ ID NO: 22 - DNA, 60 bp, Artificial Sequence
  Description of Artificial Sequence Synthetic probe
catggtatcgagtattctttatattcagttcctatttaagtcatttttgtcatgtccgcc

SEQ ID NO: 23 - DNA, 60 bp, Artificial Sequence
  Description of Artificial Sequence Synthetic probe
aagtcgataatgaggaacacacccttgttcccgtcattcacgtataaagagtggctacct

SEQ ID NO: 24 - DNA, 60 bp, Artificial Sequence
  Description of Artificial Sequence Synthetic probe
caccatgaaaacagtattgaagattggacctaacaatggaaagaattttttggtgtgtcc

SEQ ID NO: 25 - DNA, 60 bp, Artificial Sequence
  Description of Artificial Sequence Synthetic probe
taatcctgtatcttatatggatatatgtatgtgtttgcattgactgggacctctttcaca

SEQ ID NO: 26 - DNA, 60 bp, Artificial Sequence
  Description of Artificial Sequence Synthetic probe
ccattcgattacattttcttttattttcctacaatgtaagcacggttttagtcctgtagg

SEQ ID NO: 27 - DNA, 60 bp, Artificial Sequence
  Description of Artificial Sequence Synthetic probe
actgccctagaaggcaaatcgcctacgttacacagtgtaccatgtggcaaacacgcacat

SEQ ID NO: 28 - DNA, 60 bp, Artificial Sequence
  Description of Artificial Sequence Synthetic probe
caagttctacatcaaacagttcgttaactgcagacgtaaattctggatttgccaaacgtg

SEQ ID NO: 29 - DNA, 60 bp, Artificial Sequence
  Description of Artificial Sequence Synthetic probe
gggcatatggaacagagcaaccgagaactgcatacattgaaccttgtgttagcattctct

SEQ ID NO: 30 - DNA, 60 bp, Artificial Sequence
  Description of Artificial Sequence Synthetic probe
gtgtccatgtatctgtactgcaatctacaataagtccacttctgcctctgccactactgc

SEQ ID NO: 31 - DNA, 60 bp, Artificial Sequence
  Description of Artificial Sequence Synthetic probe
tctttgcagattactctgcctccaaatgcagggcctttcagagatgcattgtgattgtaa

SEQ ID NO: 32 - DNA, 60 bp, Artificial Sequence
  Description of Artificial Sequence Synthetic probe
ttcaagtatgctcttttggctgtaatgggtgcatatgtgttactaaaacgtgaatcctaa

SEQ ID NO: 33 - DNA, 60 bp, Artificial Sequence
  Description of Artificial Sequence Synthetic probe
ttttctccccttacgcactttgaaacccatgctagaaaagtgaatacatctgactgtgct

SEQ ID NO: 34 - DNA, 60 bp, Artificial Sequence
  Description of Artificial Sequence Synthetic probe
catagctgccaacagctaaagcccccagtatctcttcagtctctataccaattaaagtga

SEQ ID NO: 35 - DNA, 60 bp, Artificial Sequence
  Description of Artificial Sequence Synthetic probe
atttcttgtgggtctcctattaccagcttctaaatgaatgttgtttttgacccagtttgt

SEQ ID NO: 36 - DNA, 60 bp, Artificial Sequence
  Description of Artificial Sequence Synthetic probe
acacagatgacattgaaattcgtttctctcctcatctatcacactggagcaaaactggct

SEQ ID NO: 37 - DNA, 384 bp, Homo sapiens
atggcgtactccacagtgcagagagtcgctctggcttctgggcttgtcctggctctgtcgctgctgcccaaggccttcctgtcccgcgggaagcggcaggagccgccgccgacacctgaaggaaaattgggccgatttccacctatgatgcatcatcaccaggcaccctcagatggccagactcctggggctcgtttccagaggtctcaccttgccgaggcatttgcaaaggccaaaggatcaggtggaggtgctggaggaggaggtagtggaagaggtctgatggggcagattattccaatctacggttttgggatttttttatatatactgtacattctatttaaggtaagtagaatcatcctaaccatattacatcaatga

SEQ ID NO: 38 - DNA, 333 bp, Homo sapiens
atgcaggcggccctagaggtcaccgctcgctactgtggccgggagctggagcagtatggccagtgtgtggcggccaagccggaatcctggcagcgggactgtcactaccttaagatgagcattgcccagtgcacatcctcccacccaatcatccgccagatccgccaggcctgtgctcagccttttgaggccttcgaggagtgtcttcgacagaacgaggcagctgtgggcaactgtgcagagcatatgcgccgcttcctgcagtgcgctgagcaggtgcagccgccacgctcacctgcaactgtggaggcacagccacttcctgcctcctga

SEQ ID NO: 39 - DNA, 4770 bp, Homo sapiens
atggcaaagcaacttaaccttccagaaaatacagatgattggacaaaagaggatgtaaatcagtggttagaaagtcataagattgaccaaaaacacagggaaattttgactgaacaagacgtgaatggagcagtcttgaagtggttaaaaaaagaacatcttgttgatatgggcatcacacatggaccagctattcaaatagaagaactattcaaagaattgcggaaaacagccattgaagattcgattcagacatctaagatgggaaagcccagtaaaaatgctcctaaagaccaaactgtgtctcaaaaggaacgtagagaaacttcaaagcaaaaacaaaagggtaaagagaacccagatatggctaatccgtctgcaatgagtacaactgctaaaggttctaagtcactaaaagttgagctcatagaagataaaatagattatacaaaggaaaggcaaccatccatagacctgacatgtgtatcatatccatttgatgaattcagtaatccatatcgttacaagttggattttagtctacagcctgaaacaggaccaggcaatctcattgatccgatacatgaattcaaagccttcacaaatacagcaacagccacagaagaggatgtcaagatgaaatttagcaatgaggttttccgatttgcttcagcttgtatgaattcacgtaccaatggcactattcattttggagtcaaagacaaaccccatgggaaaattgttggcatcaaagtcaccaatgataccaaggaagccctcattaaccatttcaatctgatgataaacaagtattttgaagaccatcaagtccaacaagcaaagaagtgcattcgagagccaagatttgtggaagttttactgccaaatagtactctatctgacagatttgttattgaagtggacattattccacagttctctgaatgccaatatgattatttccagattaaaatgcaaaattacaacaacaaaatatgggaacaaagtaaaaaattctcactatttgtgcgagatgggaccagctctaaggacattacgaaaaataaagttgatttcagagcatttaaagcagattttaaaacactggcagagtccagaaaagcagcagaagaaaaattcagagcaaaaacaaataaaaaagaaagagagggaccaaagttggttaaattattgacaggaaatcaagatttgttagataattcatactatgaacagtacattcttgtaacaaataaatgccacccagatcaaacaaaacacttagatttcctgaaggaaattaaatggtttgctgtattggagtttgatcctgagtctaacatcaatggagtggtcaaagcttacaaagaaagccgagtagcaaaccttcactttccaagtgtatatgtagaacagaaaaccacaccaaatgagacgatttctactctaaatctttaccatcaacccagctggattttctgcaatggcaggttagaccttgacagtgaaaaatataaaccctttgatccaagttcctggcaaagagaaagagcttctgatgtcaggaaactgatttcatttcttacacatgaagacataatgccaagagggaagtttttggtggtatttctattactgtcctctgtggatgacccaagagatcccctcattgagactttctgtgctttctaccaggatctcaaaggaatggaaaatatactgtgtatttgtgtgcacccacacatatttcagggatggaaagatctacttgaagcaagattaataaaacaccaagatgaaatttcaagccaatgtatttctgctttaagccttgaagagatcaatggcactattcttaaactaaaatctgtgactcaatcttcaaaaaggcttttgccatctattggtttatcgactgtccttctgaaaaaggaagaagatatcatgactgctctggaaattatctgtgaaaatgaatgtgagggtacactgttagagaaggacaaaaataaattccttgaattcaaggcatcaaaagaggaagacttctatcgaggtggcaaagtgtcatggtggaacttctacttctcttctgaaagttattcttcaccttttgtcaaaagggataaatatgaaagacttgaagcaatgattcaaaactgtgcagattcttctaaaccaacaagtaccaaaattattcatctgtatcatcatccaggctgtgggggaactaccttggctatgcacattctctgggaactaaggaagaaattcagatgtgctgtgctgaaaaacaagacagtggatttttctgaaattggagaacaggtaaccagtttaatcacctatggggcaatgaaccgtcaggaatacgtacctgtactactccttgttgatgattttgaagaacaagataatgtctatcttctgcagtactctattcaaacagctatagctaaaaagtacattcgatatgaaaaacctctggtgattatcctaaattgtatgagatcacaaaatcctgaaaaaagtgcaaggatcccagacagtattgccgtaatacagcaactctctcccaaagaacagagagcttttgagcttaaattgaaagaaatcaaagaacagcataaaaactttgaggatttttattcctttatgatcatgaaaaccaattttaataaagaatacatagaaaatgtggtccggaatatcctgaaagggcagaatattttcaccaaggaagcaaagctcttttcttttctggctcttcttaattcatatgtgcctgataccaccatttcactatcacagtgtgaaaaattcttaggaattggaaacaagaaggctttctgggggacagaaaaatttgaagacaagatgggcacctactctacaattctgataaaaacagaggtcatcgaatgtgggaactactgtggagtacgcatcattcactctttgattgcagagttctcactggaagaattgaagaaaagctatcacctgaataaaagtcaaattatgttggatatgctaactgagaatttgttcttcgatactggtatgggaaaaagtaaatttttgcaagatatgcacacactcctactcacaagacaccgcgatgaacatgaaggtgaaacaggaaattggttttccccatttattgaagcattacataaagatgaaggaaatgaagcagttgaagctgtattgcttgaaagtatccatcggttcaacccaaatgcattcatttgccaagcgttggcaagacatttctacattaaaaagaaggactttggcaatgctctaaactgggcaaaacaagcaaaaatcatagaacctgacaattcttatatctcagatacactgggtcaagtctacaaaagtaaaataagatggtggatagaggaaaacggaggaaacgggaacatttcagttgatgatctaattgctcttttggatttagcagaacatgcctcaagtgcattcaaagaatctcaacagcaaagtgaagatagagagtatgaagtgaaggaaagattgtatccgaagtcaaaaaggcggtatgatacttacaatatagctggttatcaaggagagatagaagttgggctttacacaatccaaattctccagctcattcctttttttgataataaaaatgagctatctaaaagatatatggtcaattttgtatcaggaagtagtgatattccaggggatccaaacaatgaatataaattagccctcaaaaactatattccttatttaactaaattgaaattttctttgaaaaagtcctttgatttttttgatgaatactttgtcctgctaaaacccaggaacaatattaagcaaaatgaagaggccaaaactcggagaaaggtggctggatattttaagaaatatgtagatatattttgtctcttagaagaatcacaaaacaacacaggtcttggatcaaagttcagtgagccacttcaagtagagagatgcaggagaaacctagtagctttaaaagcagacaagttttctgggctcttggaatatcttatcaaaagtcaagaggatgctataagcactatgaaatgtatagtgaacgaatatacttttctcttagaacaatgcactgtcaaaatccagtcaaaagaaaagctaaatttcatcttggccaacattattctctcctgtatccaacctacctccagattagtaaagccagttgaaaaactaaaagatcagcttcgagaagtcttgcaaccaataggactgacttatcagttttcagaaccgtattttctagcttccctcttattctggccagaaaatcaacaactagatcaacattctgaacaaatgaaagagtatgctcaagcactaaaaaattctttcaaggggcaatataaacatatgcatcgtacaaagcaaccaattgcatatttctttcttggaaaaggtaaaagactggaaagacttgttcacaaaggaaaaattgaccagtgctttaagaagacaccagatattaattccttgtggcagagtggagatgtgtggaaggaggaaaaagtccaagaacttttgcttcgtttacaaggtcgagctgaaaacaattgtttatatatagaatatggaatcaatgaaaaaatcacaatacccatcactcccgcttttttaggtcaacttagaagtggcagaagcatagagaaggtgtctttttacctgggattttccattggaggcccacttgcttatgacattgaaattgtttaa

SEQ ID NO: 40 - DNA, 702 bp, Homo sapiens
atggcggccaccagtggaactgatgagccggtttccggggagttggtgtctgtggcacatgcgctttctctcccagcagagtcgtatggcaacgatcctgacattgagatggcttgggccatgagagcaatgcagcatgctgaagtctattacaagctgatttcatcagttgacccacagttcctgaaactcaccaaagtagatgaccaaatttactctgagttccggaaaaattttgagacccttaggatagatgtgttggacccagaagaactcaagtcagaatcagccaaagagaagtggaggccattctgcttgaagtttaatgggattgttgaagacttcaactatggtactttgctgcgactagattgttctcagggctacactgaggaaaacaccatctttgcccccaggatacaattctttgccattgaaattgctcggaaccgggaaggctataacaaagctgtttatatcagtgttcaggacaaagaaggagagaaaggagtcaacaatggaggagaaaaaagagctgacagtggagaagaagagaacaccaagaatggaggagagaaaggagctgatagtggagaagaaaaagaggaaggaatcaacagagaagacaaaactgacaaaggaggagaaaaagggaaagaagctgacaaagaaatcaacaaaagtggtgaaaaagctatgtaa

SEQ ID NO: 41 - DNA, 1563 bp, Homo sapiens
atggcctcggaccccatcttcacgctggcgcccccgctgcattgccactacggggccttcccccctaatgcctctggctgggagcagcctcccaatgccagcggcgtcagcgtcgccagcgctgccctagcagccagcgccgccagccgtgtcgccaccagtaccgacccctcgtgcagcggcttcgccccgccggacttcaaccattgcctcaaggattgggactataatggccttcctgtgctcaccaccaacgccatcggccagtgggatctggtgtgtgacctgggctggcaggtgatcctggagcagatcctcttcatcttgggctttgcctccggctacctgttcctgggttaccccgcagacagatttggccgtcgcgggattgtgctgctgaccttggggctggtgggcccctgtggagtaggaggggctgctgcaggctcctccacaggcgtcatggccctccgattcctcttgggctttctgcttgccggtgttgacctgggtgtctacctgatgcgcctggagctgtgcgacccaacccagaggcttcgggtggccctggcaggggagttggtgggggtgggagggcacttcctgttcctgggcctggcccttgtctctaaggattggcgattcctacagcgaatgatcaccgctccctgcatcctcttcctgttttatggctggcctggtttgttcctggagtccgcacggtggctgatagtgaagcggcagattgaggaggctcagtctgtgctgaggatcctggctgagcgaaaccggccccatgggcagatgctgggggaggaggcccaggaggccctgcaggacctggagaatacctgccctctccctgcaacatcctccttttcctttgcttccctcctcaactaccgcaacatctggaaaaatctgcttatcctgggcttcaccaacttcattgcccatgccattcgccactgctaccagcctgtgggaggaggagggagcccatcggacttctacctgtgctctctgctggccagcggcaccgcagccctggcctgtgtcttcctgggggtcaccgtggaccgatttggccgccggggcatccttcttctctccatgacccttaccggcattgcttccctggtcctgctgggcctgtgggattatctgaacgaggctgccatcaccactttctctgtccttgggctcttctcctcccaagctgccgccatcctcagcaccctccttgctgctgaggtcatccccaccactgtccggggccgtggcctgggcctgatcatggctctaggggcgcttggaggactgagcggcccggcccagcgcctccacatgggccatggagccttcctgcagcacgtggtgctggcggcctgcgccctcctctgcattctcagcattatgctgctgccggagaccaagcgcaagctcctgcccgaggtgctccgggacggggagctgtgtcgccggccttccctgctgcggcagccaccccctacccgctgtgaccacgtcccgctgcttgccacccccaaccctgccctctga

SEQ ID NO: 42 - DNA, 1590 bp, Homo sapiens
atggagcaagaaaaaaaactgttggtctcagattctaacagctttatggagagggagagtttgaaaagccctttcacaggagatacaagtatgaataatttggaaactgttcaccacaataattctaaggcagataaacttaaagagaaaccttcagaatggtctaaaagacatagaccacaacattataagcatgaggatgcaaaagaaatgccactgacatgggttcaagatgagatttggtgtcatgattcctatgagagtgatggcaagtcagagaattggggaaattttatagctaaagaggaggaaaaacccaatcaccaggaatgggactcaggagaacataccaatgcctgtgtccagcagaattcatcctttgtagacagaccctataaatgttccgaatgttggaaaagcttcagtaatagttctcatttgcgtactcaccagaggacccactcaggagaaaagccttataaatgctctgagtgtgcaaaatgtttttgtaacagttctcacctgattcagcatctaagaatgcacacaggagagaagccctaccagtgtggtgaatgtgggaaaagcttcagcaatacctcccatcttattatccatgagagaactcacacgggagagaaaccctacaaatgtcccgagtgtgggaagagattcagcagcagctctcaccttattcagcatcacagatcacatacaggtgaaaaaccatatgaatgttctgtctgcggaaaaggcttcagtcacagctatgtcctaatagaacatcagaggactcacactggagaaaaaccttataagtgccctgattgtgggaagagttttagtcagagttccagcctcattcgccaccagcggacacacacaggtgagaagccctacaaatgtcttgagtgtgaaaaaagctttggttgtaattctactctaataaaacatcagagaatacatacaggagaaaagccttatcaatgtccagaatgtgggaagaattttagtcgtagttcaaaccttattacacaccagaaaatgcacacaggagagaaatcctatgaaagttctgaatatgaggaaagtttgggtcagaactgcaatgtgatagaagaatgcagaatccagttaggagagaaaccatatagatgttgtgaatgtgggaagagttttggccttagctcccatctcattagacatcagagaacacatacaggagaaaaaccttacagatgttctgagtgctggaaaactttcagtcagagttccaccctggtgattcaccaaaggacacatacaggagagaaaccttataaatgtcctgattgtggtgaaagcttcagtcagagctttaaccttatcaggcaccggaggacccacataggggaaaaaccttacaaatgtaccagctgtgagaaatgcttcagcagaattgcctacctcagtcagcatcggaaaattcacgtagaaaagccttttgagtctcccgacgttggggattttcctcatgaatggacttggaaaaactgttcaggggaaatgcccttcatctcttcattttccgtctcaaattcatcttcctga

SEQ ID NO: 43 - DNA, 903 bp, Homo sapiens
atgcagctcatcaaccaccccaatctcaacacgatggatgagttctgtgaacaagttggaaaagggaccgaaaacaacgtcggcaaaggccaggacataccccagggaacagtaacaaatggaagcacttacaagaaaataagaaaggtagaaacaggaggacccacgaatttgagagggcacccaggagtgtctgagctgcattacctctagaaacctcaaacaagtagaaacttgcctagacaataactggaaaaacaaatgcaatatacatgaacttttttcatggcattatgtggatgtttacaatggtgggaaattcagctgagttccaccaattataaattaaatccatgagtaactttcctaataggctttttttcctaataccaccacctaacagagaacacaggtgaatgcagatgttcactttagcagacttaatgtttcctatgagatttcactgtacaggtttgtctttcttctttgcctgagaaataaaaatgtcatttgccatattgccatctaaaggagaaaaactgcatcagcaaagccattgtattgaactaaaagtttaaaatgaactgcatggatttactaagctgatgaatattccaaaacgtggttggattcaaggatatattttgtctaccggccctcatgtttgtatgtacttgaggagtaaaatgagtaaaatgatactgaatgaaatgttctgtggaaatattaaaaaaaaaaaaaaacataagccatccatcatccagaagaaaaatggaatacactgatctactactgatgtcttctttcagctttgatctaaagatgtattttattaaaactataatttaaatgtaccatgaaaaatatgcagtaaaaattagttgttttctaagctagagtagg

SEQ ID NO: 44 - DNA, 4173 bp, Homo sapiens
atggtggttctccgcagcagcttggagctgcacaaccactccgcggcctcggccacgggctccttggacctgtccagtgacttcctcagtctggagcacatcggccggaggcggctccgctcggccggcgcggcgcagaagaaacccgcggcgaccacagccaaagcgggcgatgggtcatcagttaaggaagttgaaacctaccaccggacacgtgctttaagatctttgagaaaagatgcacagaattcttcagattctagttttgagaagaatgtggaaataacggagcaacttgctaatggcaggcattttacaaggcagttggccagacagcaggctgataaaaaaaaagaagagcacagagaagacaaagtgattccagttactcggtcattgagggctagaaacatcgttcaaagtacagaacacttacatgaagataatggtgatgttgaagtgcgtcgaagttgtaggattagaagtcgttatagtggtgtaaaccagtccatgctgtttgacaaacttataactaacactgctgaagctgtacttcaaaaaatggatgacatgaagaagatgcgtagacagcgaatgagagaacttgaagacttgggagtgtttaatgaaacagaagaaagcaatcttaatatgtacacaagaggaaaacagaaagatattcaaagaactgatgaagaaacaactgataatcaagaaggcagtgtggagtcatctgaagagggtgaagaccaagaacatgaagatgatggtgaagatgaagatgatgaagatgatgatgatgatgacgatgatgatgatgatgatgatgatgaagatgatgaagatgaagaagatggagaagaagagaatcagaagcgatattatcttagacagagaaaagctactgtttactatcaggctccattggaaaaacctcgtcaccagagaaagcccaacatattttatagtggcccagcttctcctgcaagaccaagataccgattatcttccgcaggaccaagaagtccttactgtaaacgaatgaacaggcgaaggcatgcaatccacagtagtgactcgacttcatcttcctcctctgaagatgaacagcactttgagaggcggaggaaaaggagtcgtaatagggctatcaataggtgcctcccactaaattttcggaaagatgaattaaaaggcatttataaagatcgaatgaaaattggagcaagccttgccgatgttgatccaatgcaactagattcttcagtacgatttgatagtgttggtggcctgtctaatcatatagcagctctaaaagagatggtggtgtttccattactttatccagaagtctttgaaaaatttaaaattcaacccccaagaggttgtttgttttatgggccacctggaactggaaagactctggttgccagagcacttgccaatgagtgcagtcaaggggataaaagagtagcatttttcatgaggaaaggtgctgattgtctaagtaaatgggtaggagaatctgaaagacagctacgattgctgtttgatcaggcctatcagatgcgcccatcaattattttttttgacgaaattgatggtctggctccagtacggtcaagcaggcaagatcagattcacagttctattgtttccaccctgctagctcttatggatggattggacagcagaggggaaattgtggtcattggtgctacgaacaggctagattctatagatcctgctttacgaaggcctggtcgctttgatagagaattcctctttagcctgcctgataaagaggctcgaaaagagattctaaagattcacaccagggattggaatcccaaaccactggacacatttttagaagagctagcagaaaactgtgttggatactgtggagcagatattaaatcaatatgtgctgaagctgctttatgtgctttacgacgacgctacccacagatctataccactagtgagaaactgcagttggatctctcttcaattaatatctcagctaaggatttcgaggtagctatgcaaaagatgataccagcctcccaaagagctgtgacatcacctgggcaggcactgtccaccgttgtgaaaccactcctgcaaaacactgttgacaagattttagaagccctgcagagagtatttccacatgcagaattcagaacaaataaaacattagactcagatatttcttgtcctctgctagaaagtgacttggcttacagtgatgatgatgttccatcagtttatgaaaatggactttctcagaaatcttctcataaggcaaaagacaattttaattttcttcatttgaatagaaatgcttgttaccaacctatgtcttttcgaccaagaatattgatagtaggagaaccaggatttgggcaaggttctcacttggcaccagctgtcattcatgctttggaaaagtttactgtatatacattagacattcctgttctttttggagttagtactacatcccctgaagaaacatgtgcccaggtgattcgtgaagctaagagaacagcaccaagtatagtgtatgttcctcatatccacgtgtggtgggaaatagttggaccgacacttaaagccacatttaccacattattacagaatattccttcatttgctccagttttactacttgcaacttctgacaaaccccattccgctttgccagaagaggtgcaagaattgtttatccgtgattatggagagatttttaatgtccagttaccggataaagaagaacggacaaaattttttgaagatttaattctaaaacaagctgctaagcctcctatatcaaaaaagaaagcagttttgcaggctttggaggtactcccagtagcaccaccacctgagccaagatcactgacagcagaagaagtgaaacgactagaagaacaagaagaagatacatttagagaactgaggattttcttaagaaatgttacacataggcttgctattgacaagcgattccgagtgtttactaagcctgttgaccctgatgaggttcctgattatgtcactgtaataaagcaaccaatggacctttcatctgtaatcagtaaaattgatctacacaagtatctgactgtgaaagactatttgagagatattgatctaatctgtagtaatgccttagaatacaatccagatagagatcctggagatcgtcttattaggcatagagcctgtgctttaagagatactgcctatgccataattaaagaagaacttgatgaagactttgagcagctctgtgaagaaattcaggaatctagaaagaaaagaggttgtagctcctccaaatatgccccgtcttactaccatgtgatgccaaagcaaaattccactcttgttggtgataaaagatcagacccagagcagaatgaaaagctaaagacaccgagtactcctgtggcttgcagcactcctgctcagttgaagaggaaaattcgcaaaaagtcaaactggtacttaggcaccataaaaaagcgaaggaagatttcacaggcaaaggatgatagccagaatgccatagatcacaaaattgagagtgatacagaggaaactcaagacacaagtgtagatcataatgagaccggaaacacaggagagtcttcggtggaagaaaatgaaaaacagcaaaatgcctctgaaagcaaactggaattgagaaataattcaaatacttgtaatatagagaatgagcttgaagactctaggaagactacagcatgtacagaattgagagacaagattgcttgtaatggagatgcttctagctctcagataatacatatttctgatgaaaatgaaggaaaagaaatgtgtgttctgcgaatgactcgagctagacgttcccaggtagaacagcagcagctcatcactgttgaaaaggctttggcaattctttctcagcctacaccctcacttgttgtggatcatgagcgattaaaaaatcttttgaagactgttgttaaaaaaagtcaaaactacaacatatttcagttggaaaatttgtatgcagtaatcagccaatgtatttatcggcatcgcaaggaccatgataaaacatcacttattcagaaaatggagcaagaggtagaaaacttcagttgttccagatga

SEQ ID NO: 45 - DNA, 930 bp, Homo sapiens
atgaggcaaaatgacaaaatcatgtgcatattggaaaaccggaaaaagagggataggaaaaatctctgtagggctatcaatgacttccaacagagctttcagaagccagaaactcgccgtgaatttgatctgtccgaccccctagcccttaagaaagatcttccagcccggcagtcagataatgatgttcggaatacgatatcaggaatgcagaaattcatgggagaggatttaaacttccatgagaggaagaaattccaagaggaacaaaacagagaatggtctttgcagcagcaaagggaatggaagaacgcccgtgctgaacaaaaatgcgcagaggccctctacacagagacaaggctgcagtttgacgagacagccaagcacctccagaagctggaaagcaccaccagaaaggcagtttgtgcatctgtgaaagacttcaacaagagccaggccatcgagtcagtggaaaggaaaaagcaagagaaaaagcaagaacaagaggacaacttggccgagatcaccaacctcctgcgtggggacctgctctccgagaacccgcagcaggcagccagctccttcgggccccaccgcgtggtccctgaccgctggaagggcatgacccaggagcagctggagcagatccgcctagtccagaagcagcaaatccaggagaagctgaggctccaggaagaaaagcgccagcgagacctggactgggaccggcggaggattcagggggctcgcgccaccctgctgtttgagcggcagcagtggcggcggcagcgcgacctgcgcagagctctggacagcagcaacctcagcctggccaaggagcagcatttgcagaaaaaatatatgaatgaagtctatacaaatcaacccacgggagactatttcacacaatttaatacaggaagtcgataa

SEQ ID NO: 46 - DNA, 1818 bp, Homo sapiens
atggtggaaggaccaggctgtactctgaatggagagaagattcgcgcgcgggtgctcccgggccaggcggtgaccggcgtgcggggaagcgctctgcggagtccgcagggccgcgccttgcggctcgcagcctccacggttgtggtctccccgcaggctgctgcactgaataatgattccagccagaatgtcttgagcctgtttaatggatatgtttacagtggcgtggaaactttggggaaggagctctttatgtactttggaccaaaagctttacggattcatttcggaatgaaaggcttcatcatgattaatccacttgagtataaatataaaaatggagcttctcctgttttggaagtgcagctcaccaaagatttgatttgtttctttgactcatcagtagaactcagaaactcaatggaaagccaacagagaataagaatgatgaaagaattagatgtatgttcacctgaatttagtttcttgagagcagaaagtgaagttaaaaaacagaaaggccggatgctaggtgatgtgctaatggatcagaacgtattgcctggagtagggaacatcatcaaaaatgaagctctctttgacagtggtctccacccagctgttaaagtttgtcaattaacagatgaacagatccatcacctcatgaaaatgatacgtgatttcagcattctcttttacaggtgccgtaaagcaggacttgctctctctaaacactataaggtttacaagcgtcctaattgtggtcagtgccactgcagaataactgtgtgccgctttggggacaataacagaatgacatatttctgtcctcactgtcaaaaagaaaatcctcaacatgttgacatatgcaagctaccgactagaaatactataatcagttggacatctagcagggtggatcatgttatggactccgtggctcggaagtcggaagagcactggacctgtgtggtgtgtactttaatcaataagccctcttctaaggcatgtgatgcttgcttgacctcaaggcctattgattcagtgctcaagagtgaagaaaattctactgtctttagccacttaatgaagtacccgtgtaatacttttggaaaacctcatacagaagtcaagatcaacaggaaaactgcatttggaactacaactcttgtcttgactgattttagcaataaatccagtactttggaaagaaaaacaaagcaaaaccagatactagatgaggagtttcaaaactctcctcctgctagtgtgtgtttgaatgatatacagcacccctccaagaagacaacaaacgatataactcaactatccagcaaagtaaacatatcacctacaatcagttcagaatctaaattatttagtccagcacataaaaaaccgaaaacagcccactactcatcaccagagcttaaaagctgcaaccctggatattctaacagtgaacttcaaattaatatgacagatggccctcgtaccttaaatcctgacagccctcgctgcagtaaacacaaccgcctctgcatcctccgagttgtgaggaaggatggggaaaacaagggcaggcagttttatgcctgtcctctacctagagaagcacaatgtggattttttgaatgggcagatttgtccttcccattctgcaaccatggcaagcgttccaccatgaaaacagtattgaagattggacctaacaatggaaagaatttttttgtgtgtcctcttgggaaggaaaaacaatgcaattttttccagtgggcagaaaatgggccaggaataaaaattattcctggatgctaa

SEQ ID NO: 47 - DNA, 624 bp, Homo sapiens
atgagtgaggcggttcgggtaccctcgcccgccactccgctggtggtggcggcggccgcgcctgaggagaggaaagggaaggagtcagagcgcgagaagctgccgcccatcgtatcggcgggcgccggcgcgaccgcgggtttggacagaggagccaaaggccaaatttccactttcagcagttttatttcagctgttagcccgaagaaagaagctgctgaaaacagaagttcacctgcacatcttgttttccctaacatcaagaatgtgagagaaccaccaccaatttgccttgatgttagacaaaaacagcgtacatctatggatgcatcatcatccgaaatgaaggccccagtccttccagaacctattcttcctatccagcccaaaactgtgaaagactttcaggaagatgtagaaaaagttaagtcatcaggagattggaaagcagtacatgatttttatctaacaacgtttgattctttcccagaattaaatgctgcatttaagaaagatgccactgcctcatttaacaccattgaagactctgggattaatgctaaatttgtgaatgctgtgtatgataccttacttaatactgtaagtattatgacatgcaagtaa

SEQ ID NO: 48 - DNA, 465 bp, Homo sapiens
atgtcgcgggtgagtggtgtcgaggcctgttgggtcagggcggttcgcaggtgctgtcagagctgggcggggcagccgggtcgtagaggcgggcgccagtcgcggtcggtggagagggatgaggatgtaggagggacggacgtggcggaagccgcggggactgcgggggcggagtgcctctggggagccagggaggcctttccagaggctcctgggggaagaagaggcgaagcgagagtccccggggaagccctactccacccccagccggagcctgggttgtgcctgcacggaccagagcccacagtgcgagttgctgtaggcaaccagctagggtggccaacttctcccggttggcccgagatgttctggttttgggaccacagcgtcccaggcctccagcccaccacagtgaccatattgtgcgccccctgcccatcttctcccgcagcttccctagattag

SEQ ID NO: 49 - DNA, 705 bp, Homo sapiens
atgtcggtgaaggagggcgcacagcgcaagtgggcagcgctgaaggagaagctggggccacaggattcggaccccacggaggccaacctggagagcgcggaccctgagctgtgcatccggctgctccagatgccctctgtggtcaactactccggcctgcgcaagcgcctggagggcagcgacggcggctggatggtgcagttcctggagcagagcggcctggacctgctgctggaggcgctggcgcggctgtcgggccgcggcgttgcacgtatctccgacgccctgctgcagctcacctgcgtcagctgcgtgcgcgccgtcatgaactcgcggcagggcatcgagtacatcctcagcaaccagggctacgtgcgccagctctcccaggccctggacacatccaacgtgatggtgaagaagcaggtgtttgagctactggctgccctgtgcatctactctcccgagggccacgtgctgaccctggacgccctggaccactacaagacggtgtgcagccagcagtaccgcttcagcattgtcatgaacgagctctccggcagcgacaacgtgccctacgtggtcaccctgcttagcgtgatcaacgccgtcatcttgggccccgaggacctgcgcgcgcgcacccagctgcggaacgagtttatcgggctgcagctgctggacgtcctggctcgcctgcggtga

SEQ ID NO: 50 - DNA, 2697 bp, Homo sapiens
atgtctgtcactgaggaagacctgtgccaccatatgaaagtagtagttcgtgtacgtccggaaaacactaaagaaaaagcagctggatttcataaagtggttcatgttgtggataaacatatcctagtttttgatcccaaacaagaagaagtcagttttttccatggaaagaaaactacaaatcaaaatgttataaagaaacaaaataaggatcttaaatttgtatttgatgctgtttttgatgaaacgtcaactcagtcagaagtttttgaacacactactaagccaattcttcgtagttttttgaatggatataattgcacagtacttgcctatggtgccactggtgctgggaagacccacactatgctaggatcagctgatgaacctggagtgatgtatctaacaatgttacacctttacaaatgcatggatgagattaaagaagagaaaatatgtagtactgcagtttcatatctggaggtatataatgaacagattcgtgatctcttagtaaattcagggccacttgctgtccgggaagatacccaaaaaggggtggtcgttcatggacttactttacaccagcccaaatcctcagaagaaattttacatttattggataatggaaacaaaaacaggacacaacatcccactgatatgaatgccacatcttctcgttctcatgctgttttccaaatttacttgcgacaacaagacaaaacagcaagtatcaatcaaaatgtccgtattgccaagatgtcactcattgacctggcaggatctgagcgagcaagtacttccggtgctaaggggacccgatttgtagaaggcacaaatattaatagatcacttttagctcttgggaatgtcatcaatgccttagcagattcaaagagaaagaatcagcatatcccttacagaaatagtaagcttactcgcttgttaaaggattctcttggaggaaactgtcaaactataatgatagctgctgttagtccttcctctgtattctacgatgacacatataacactcttaagtatgctaaccgggcaaaggacattaaatcttctttgaagagcaatgttcttaatgtcaataatcatataactcaatatgtaaagatctgtaatgagcagaaggcagagattttattgttaaaagaaaaactaaaagcctatgaagaacagaaagccttcactaatgaaaatgaccaagcaaagttaatgatttcaaaccctcaggaaaaagaaatcgaaaggtttcaagaaatcctgaactgcttgttccagaatcgagaagaaattagacaagaatatctgaagttggaaatgttacttaaagaaaatgaacttaaatcattctaccaacaacagtgccataaacaaatagaaatgatgtgttctgaagacaaagtagaaaaggccactggaaaacgagatcatagacttgcaatgttgaaaactcgtcgctcctacctggagaaaaggagggaggaggaattgaagcaatttgatgagaatactaattggctccatcgtgtcgaaaaagaaatgggactcttaagtcaaaacggtcatattccaaaggaactcaagaaagatcttcattgtcaccatttgcacctccagaacaaagatttgaaagcacaaattagacatatgatggatctagcttgtcttcaggaacagcaacacaggcagactgaagcagtattgaatgctttacttccaaccctaagaaaacaatattgcacattaaaagaagccggcctgtcaaatgctgcttttgaatctgacttcaaagagatcgaacatttggtagagaggaaaaaagtggtagtttgggctgaccaaactggcgaacaaccaaagcaaaacgatctacccgggatttctgttcttatgaccttttcacaacttggaccagttcagcctattccttgttgctcatcttcaggtggaactaatctggttaagattcctacagaaaaaagaactcggagaaaactaatgccatctcccttgaaaggacagcatactctaaagtctccaccatctcaaagtgtgcagctcaatgattctcttagcaaagaacttcagcctattgtatatacaccagaagactgtagaaaagcttttcaaaatccgtctacagtaaccttaatgaaaccatcatcatttactacaagttttcaggctatcagctcaaacataaacagtgataattgtctgaaaatgttgtgtgaagtagctatccctcataatagaagaaaagaatgtggacaggaggacttggactctacatttactatatgtgaagacatcaagagctcgaagtgtaaattacccgaacaagaatcactaccaaatgataacaaagacattttacaacggcttgatccttcttcattctcaactaagcattctatgcctgtaccaagcatggtgccatcctacatggcaatgactactgctgccaaaaggaaacggaaattaacaagttctacatcaaacagttcgttaactgcagacgtaaattctggatttgccaaacgtgttcgacaagataattcaagtgagaagcacttacaagaaaacaaaccaacaatggaacataaaagaaacatctgtaaaataaatccaagcatggttagaaaatttggaagaaatatttcaaaaggaaatctaagataa

SEQ ID NO: 51 - DNA, 1869 bp, Homo sapiens
atggctgaaaacacagagggggatctgaactccaacctgctccacgccccctaccacaccggggaccctcagctggacacggccatcgggcagtggctccgctgggataagaatcccaaaacaaaagagcagattgaaaacctgttacggaatgggatgaacaaggagctgcgagatcgtctttgttgccgaatgacttttgggactgcaggacttcgttctgccatgggggcagggttttgctatattaatgaccttacagtaatacagtcaacacaggggatgtacaaataccttgagagatgtttctcagacttcaagcagagaggctttgtggttgggtatgacactcggggtcaagtaactagcagctgcagcagccagaggcttgctaaactcactgctgcagtcttgctggccaaagatgttcctgtgtaccttttttcaagatatgttcctacaccttttgtaccatatgcagttcagaagctcaaagcagttgcaggtgtgatgattactgcctctcacaaccgcaaggaagacaatggatacaaggtttactgggaaactggtgctcagatcacatctcctcatgataaagaaattctaaaatgtatagaagaatgtgtggaaccctggaatggttcctggaatgataatttagtggataccagcccgctgaagagagaccctctgcaggacatttgcaggagatacatggaagatctgaaaaagatctgtttttacagggagttaaactcgaagaccaccttgaaatttgtgcacacatcttttcatggggtcggacatgactatgtgcagttggcttttaaagtgtttggttttaagcctccaattccagtaccagaacaaaaagatcctgatccagacttttctaccgttaaatgtccaaatcctgaagaaggagaatctgtgctggaactttccttgagactggcagagaaagaaaatgcccgggtagtgctagccacagatcctgatgcagacagactggcagcagcagaacttcaggagaatggttgttggaaagttttcacagggaatgagttggcagctttgtttggatggtggatgtttgattgctggaagaaaaataaatcaagaaatgctgatgtgaagaacgtttatatgttagccaccacagtctcttctaaaattctgaaggcaattgcacttaaagaaggatttcattttgaagaaacattaccaggttttaaatggattggaagtaggataatagacctcctggaaaatgggaaagaagtcctttttgcatttgaagagtctattggttttctctgtggaacttcagttttggataaagatggggtgagtgcagctgttgtggttgctgagatggcatcttacctggaaaccatgaatataacattgaaacagcaactggttaaggtttatgaaaaatatggttatcatatttcaaaaacttcctatttcttgtgttatgaaccacctaccatcaaaagtatatttgaaaggcttcgtaattttgattctccaaaagaatatccaaaattttgtggaacatttgctatattgcatgtacgggacgttaccactggatatgacagtagccagcctaataagaaatcagtgctgcctgtgagtaaaaacagccaaatgattacatttacttttcaaaatggctgtgttgctacccttcggacaagtggaacagaaccaaagataaagtattatgcagagatgtgtgcgtcacctgaccagagtgacactgctttactggaggaagaactgaagaaactcattgatgctctgatagagaattttcttcagcctagtaagaatggactgatctggcgttctgtttag

SEQ ID NO: 52 - DNA, 726 bp, Homo sapiens
atgcagtgcttcagcttcattaagaccatgatgatcctcttcaatttgctcatctttctgtgtggtgcagccctgttggcagtgggcatctgggtgtcaatcgatggggcatcctttctgaagatcttcgggccactgtcgtccagtgccatgcagtttgtcaacgtgggctacttcctcatcgcagccggcgttgtggtctttgctcttggtttcctgggctgctatggtgctaagactgagagcaagtgtgccctcgtgacgttcttcttcatcctcctcctcatcttcattgctgaggttgcagctgctgtggtcgccttggtgtacaccacaatggctgagcacttcctgacgttgctggtagtgcctgccatcaagaaagattatggttcccaggaagacttcactcaagtgtggaacaccaccatgaaagggctcaagtgctgtggcttcaccaactatacggattttgaggactcaccctacttcaaagagaacagtgcctttcccccattctgttgcaatgacaacgtcaccaacacagccaatgaaacctgcaccaagcaaaaggctcacgaccaaaaagtagagggttgcttcaatcagcttttgtatgacatccgaactaatgcagtcaccgtgggtggtgtggcagctggaattgggggcctcgagctggctgccatgattgtgtccatgtatctgtactgcaatctacaataa

SEQ ID NO: 53 - DNA, 3390 bp, Homo sapiens
atgcctgccacacggaagccaatgagatatgggcatacagagggacacacggaggtctgttttgatgattctgggagttttattgtgacttgtggaagtgatggtgatgtgaggatttgggaagacttggatgatgatgatcctaagttcattaatgttggagaaaaggcatattcatgtgctttgaagagtggaaaactggtcactgcagtttctaataatactattcaagtccacacatttcctgaaggagttccagatggtatattgactcgcttcactacaaatgcaaaccatgtggtctttaatggggatggtactaaaattgctgctggatctagtgattttctagtcaaaattgtggatgtgatggatagcagccaacagaaaacatttcgaggacatgatgcccctgttttaagtctttcctttgatcctaaggacatctttctggcatcagctagttgtgatggatctgtcagagtgtggcaaatttcagatcagacatgtgctattagttggccactgctacaaaaatgcaacgatgtgataaatgcaaaatcaatctgcagacttgcttggcagccaaaaagtgggaagttactggcaattcctgtggaaaaatctgttaagctatatagaagagaatcttggagtcatcaatttgatctttcagataatttcatctctcagaccctcaatatagtaacctggtctccctgtgggcaatatttagctgcaggtagtattaatggtctaatcatagtttggaatgtggaaaccaaagactgcatggaaagggtgaaacatgagaaaggttatgcaatttgtggtctggcatggcatcctacttgtggtcgaatatcgtatactgatgcggaaggaaatctagggcttctagagaatgtttgtgaccccagtggaaagacatcaagcagtaaggtatctagcagagtggaaaaggattataatgatctttttgatggagatgatatgagtaatgctggtgattttctaaatgacaatgcagttgagatcccttctttttcaaaagggattataaatgatgatgaggatgatgaagacctcatgatggcttcaggtcgtcctagacagcgaagtcacatcctagaagatgatgaaaactcagttgatatttcaatgctaaaaactggttctagtcttctcaaagaggaggaggaagatggtcaagaaggcagcattcacaatctaccacttgtaacatcccaaaggccattttatgatggacccatgccaactccccggcaaaagccatttcagtcaggttctacaccgttgcatctcactcacagattcatggtgtggaactctattggaattattcgctgctataatgatgagcaagacaatgccatagatgtggagttccatgatacctccatacaccatgcaacacacttatcaaacactttgaattatacaatagcagatctttcccacgaagctattttgttggcatgtgaaagcactgatgaactagcaagcaagcttcactgcctgcactttagttcttgggattcaagcaaagagtggataatagacttgcctcagaatgaggatattgaagccatatgtctcggtcaaggatgggctgctgccgctactagtgccctgcttcttcgattgtttactattggaggggttcaaaaagaggtattcagccttgctggacctgtggtgtcaatggcaggacatggagaacagcttttcattgtttatcacagaggtacaggatttgatggggatcagtgccttggagttcaactgctagagctggggaaaaagaaaaaacaaattttgcatggtgaccctcttcctcttacaaggaaatcctaccttgcatggattgggttttcagctgaaggtaccccttgttacgtggattcagaaggaattgttcgaatgcttaacagaggacttggtaatacgtggactcctatatgtaatacaagagagcactgcaaaggaaaatctgatcactactgggtggttggtatccatgaaaatccccagcaactaaggtgcattccttgtaaaggttctcggtttcccccaacccttccacgccctgctgttgctatattatcctttaagcttccttactgtcagattgcaacagagaaaggacaaatggaggagcaattttggcgttcagttatatttcacaaccaccttgattatttagctaaaaatggttatgaatatgaagagagcactaaaaatcaagcaacaaaagagcaacaggaacttttaatgaaaatgcttgcgctttcttgtaaactggagcgagaattccgttgtgtggaacttgctgatctaatgactcaaaatgctgtgaatttagccattaaatatgcttctcgctctcggaaattaatactggctcaaaaactaagtgaactggctgtagagaaggcagccgaattgacagcaacccaggtggaagaggaagaagaagaagaagatttcagaaaaaagctgaatgctggttacagcaatactgctacagagtggagccaaccaaggttcagaaatcaagttgaagaagatgctgaggacagtggagaagctgatgatgaagaaaaaccagaaatacataagcctggacagaactcgttttccaaaagtacaaattcctctgatgtttcagctaagtcaggtgcagttacctttagcagccaaggacgagtaaatccctttaaggtatcagccagttccaaagaaccagccatgtcaatgaattcagcacgttcaactaatattttagacaatatgggcaaatcatccaagaaatccactgcacttagtcgaactacaaataatgaaaagtctcccattataaagcctctgattccaaagccgaagcctaagcaggcatctgcagcatcctatttccagaaaagaaattctcaaactaataaaactgaggaagtgaaagaagaaaatcttaaaaatgtattatctgaaaccccagctatatgtcctcctcaaaacactgaaaaccaaaggccaaagaccgggttccagatgtggttagaagaaaatagaagtaatattttgtctgacaatcctgacttttcagatgaagcagacataataaaagaaggaatgattcgatttagagtattgtcaactgaagaaagaaaggtgtgggctaacaaagccaaaggagaaacggcaagtgaaggaactgaagcaaagaagcgaaaacgtgtggttgatgaaagtgatgaaacagaaaaccaggaagaaaaagcaaaagagaacctgaatttgtctaaaaagcagaaacctttagatttttctacaaatcagaaactatcagcttttgcatttaagcaggagtaa

SEQ ID NO: 54 - DNA, 579 bp, Homo sapiens
atggtgcgtgcgggcgccgtgggggctcatctccccgcgtccggcttggatatcttcggggacctgaagaagatgaacaagcgccagctctattaccaggttttaaacttcgccatgatcgtgtcttctgcactcatgatatggaaaggcttgatcgtgctcacaggcagtgagagccccatcgtggtggtgctgagtggcagtatggagccggcctttcacagaggagacctcctgttcctcacaaatttccgggaagacccaatcagagctggtgaaatagttgtttttaaagttgaaggacgagacattccaatagttcacagagtaatcaaagttcatgaaaaagataatggagacatcaaatttctgactaaaggagataataatgaagttgatgatagaggcttgtacaaagaaggccagaactggctggaaaagaaggacgtggtgggaagagcaagagggtttttaccatatgttggtatggtcaccataataatgaatgactatccaaaattcaagtatgctcttttggctgtaatgggtgcatatgtgttactaaaacgtgaatcctaa

SEQ ID NO: 55 - DNA, 744 bp, Homo sapiens
atgaatccactcctgatccttacctttgtggcagctgctcttgctgccccctttgatgatgatgacaagatcgttgggggctacaactgtgaggagaattctgtcccctaccaggtgtccctgaattctggctaccacttctgtggtggctccctcatcaacgaacagtgggtggtatcagcaggccactgctacaagtcccgcatccaggtgagactgggagagcacaacatcgaagtcctggaggggaatgagcagttcatcaatgcagccaagatcatccgccacccccaatacgacaggaagactctgaacaatgacatcatgttaatcaagctctcctcacgtgcagtaatcaacgcccgcgtgtccaccatctctctgcccaccgcccctccagccactggcacgaagtgcctcatctctggctggggcaacactgcgagctctggcgccgactacccagacgagctgcagtgcctggacgctcctgtgctgagccaggctaagtgtgaagcctcctaccctggaaagattaccagcaacatgttctgtgtgggcttccttgagggaggcaaggattcatgtcagggtgattctggtggccctgtggtctgcaatggacagctccaaggagttgtctcctggggtgatggctgtgcccagaagaacaagcctggagtctacaccaaggtctacaactatgtgaaatggattaagaacaccatagctgccaatagctaa

SEQ ID NO: 56 - DNA, 387 bp, Homo sapiens
atggagtccaaagaggaacgagcgttaaacaatctcatcgtggaaaatgtcaaccaggaaaatgatgaaaaagatgaaaaggagcaagttgctaataaaggggagcccttggccctacctttgaatgttagtgaatactgtgtgcctagaggaaaccgtaggcggttccgcgttaggcagcccatcctgcagtatagatgggacataatgcataggcttggagagccacaggcaaggatgagagaggagaatatggaaaggattggggaggaggtgagacagctgatggaaaagctgagggaaaagcagttgagtcatagtttgcgggcagtcagcactgatccccctcaccatgaccatcacgatgagttttgccttatgccctga

SEQ ID NO: 57 - DNA, 1638 bp, Homo sapiens
ccaaggtgtgcagggagatctagccctgtgaggaagcggcacggtggccgcagggcaggaggtaaggacaccctggtctctgtgcctaggtccgtgcaagacagcggccagggcggccgggagaagctggagctcgtcctgtcgaacctgcaggcagacgtcctggagttgctgctggagtttgtctacacgggctccctggtcatcgactcggccaacgccaagacactgctggaggcggccagcaagttccagttccacaccttctgcaaagtctgcgtgtcctttctcgagaagcagctgacggccagcaactgcctgggcgtgctggccatggccgaggccatgcagtgcagcgagctctaccacatggccaaggccttcgcgctgcagatcttccccgaggtggccgcccaggaggagatcctcagcatctccaaggacgacttcatcgcctacgtctccaacgacagcctcaacaccaaggctgaggagctggtgtacgagacagtcatcaagtggatcaagaaggaccccgcgacacgcacacagtacgcggctgagctcctggccgtggtccgcctccccttcatccaccccagctacctgctcaatgtggttgacaatgaagagctgatcaagtcatcagaagcctgccgggacctggtgaacgaggccaaacgctaccatatgctgccccacgcccgccaggagatgcagacgccccgaacccggccgcgcctctctgcaggtgtggctgaggtcatcgtcttggttgggggccgtcagatggtggggatgacccagcgctcgctggtggctgtcacctgctggaacccgcagaacaacaagtggtaccccttggcctcgctgcccttctatgaccgcgagttcttcagtgtagtgagtgcaggggacaacatctacctctcaggtggaatggaatcaggggtgacgctggctgatgtctggtgctacatgtccctgcttgataactggaacctcgtctccagaatgacagtcccccgctgtcggcacaatagcctcgtctacgatgggaagatttacaccctcgggggacttggcgtggcaggcaacgtggaccacgtggagaggtacgacaccatcaccaaccaatgggaggcggtggcccctctgcccaaggcagtacactctgctgcagccacagtgtgtggcggcaagatctacgtgtttggtggggtgaacgaggcaggccgagctgccggcgtcctccagtcttacgttcctcagaccaacacgtggagcttcatcgagtccccaatgattgacaacaagtatgcccccgctgtcacgctcaatggcttcgttttcatcctgggcggggcttatgccagagctaccaccatctacgaccctgagaaaggaaacattaaggcgggcccaaacatgaaccactctcgccagttctgcagtgctgtggtgcttgatggcaagatttatgcaactggaggtattgtcagcagtgaagggcccgcgctgggcaacatggaggcctacgagcccacaaccaacacatggaccctcctcccccacatgccctgccctgtgttcagacacggctgcgtcgtgataaagaaatatattcaaagcggctga

SEQ ID NO: 58 - protein, 127 aa, Homo sapiens [sequence omitted]

SEQ ID NO: 59 - protein, 110 aa, Homo sapiens
MQAALEVTARYCGRELEQYGQCVAAKPESWQRDCHYLKMSIAQCTSSHPIIRQIRQACAQPFEAFEECLRQNEAAVGNCAEHMRRFLQCAEQVQPPRSPATVEAQPLPAS

SEQ ID NO: 60 - protein, 1589 aa, Homo sapiens
MAKQLNLPENTDDWTKEDVNQWLESHKIDQKHREILTEQDVNGAVLKWLKKEHLVDMGITHGPAIQIEELFKELRKTAIEDSIQTSKMGKPSKNAPKDQTVSQKERRETSKQKQKGKENPDMANPSAMSTTAKGSKSLKVELIEDKIDYTKERQPSIDLTCVSYPFDEFSNPYRYKLDFSLQPETGPGNLIDPIHEFKAFTNTATATEEDVKMKFSNEVFRFASACMNSRTNGTIHFGVKDKPHGKIVGIKVTNDTKEALINHFNLMINKYFEDHQVQQAKKCIREPRFVEVLLPNSTLSDRFVIEVDIIPQFSECQYDYFQIKMQNYNNKIWEQSKKFSLFVRDGTSSKDITKNKVDFRAFKADFKTLAESRKAAEEKFRAKTNKKEREGPKLVKLLTGNQDLLDNSYYEQYILVTNKCHPDQTKHLDFLKEIKWFAVLEFDPESNINGVVKAYKESRVANLHFPSVYVEQKTTPNETISTLNLYHQPSWIFCNGRLDLDSEKYKPFDPSSWQRERASDVRKLISFLTHEDIMPRGKFLVVFLLLSSVDDPRDPLIETFCAFYQDLKGMENILCICVHPHIFQGWKDLLEARLIKHQDEISSQCISALSLEEINGTILKLKSVTQSSKRLLPSIGLSTVLLKKEEDIMTALEIICENECEGTLLEKDKNKFLEFKASKEEDFYRGGKVSWWNFYFSSESYSSPFVKRDKYERLEAMIQNCADSSKPTSTKIIHLYHHPGCGGTTLAMHILWELRKKFRCAVLKNKTVDFSEIGEQVTSLITYGAMNRQEYVPVLLLVDDFEEQDNVYLLQYSIQTAIAKKYIRYEKPLVIILNCMRSQNPEKSARIPDSIAVIQQLSPKEQRAFELKLKEIKEQHKNFEDFYSFMIMKTNFNKEYIENVVRNILKGQNIFTKEAKLFSFLALLNSYVPDTTISLSQCEKFLGIGNKKAFWGTEKFEDKMGTYSTILIKTEVIECGNYCGVRIIHSLIAEFSLEELKKSYHLNKSQIMLDMLTENLFFDTGMGKSKFLQDMHTLLLTRHRDEHEGETGNWFSPFIEALHKDEGNEAVEAVLLESIHRFNPNAFICQALARHFYIKKKDFGNALNWAKQAKIIEPDNSYISDTLGQVYKSKIRWWIEENGGNGNISVDDLIALLDLAEHASSAFKESQQQSEDREYEVKERLYPKSKRRYDTYNIAGYQGEIEVGLYTIQILQLIPFFDNKNELSKRYMVNFVSGSSDIPGDPNNEYKLALKNYIPYLTKLKFSLKKSFDFFDEYFVLLKPRNNIKQNEEAKTRRKVAGYFKKYVDIFCLLEESQNNTGLGSKFSEPLQVERCRRNLVALKADKFSGLLEYLIKSQEDAISTMKCIVNEYTFLLEQCTVKIQSKEKLNFILANIILSCIQPTSRLVKPVEKLKDQLREVLQPIGLTYQFSEPYFLASLLFWPENQQLDQHSEQMKEYAQALKNSFKGQYKHMHRTKQPIAYFFLGKGKRLERLVHKGKIDQCFKKTPDINSLWQSGDVWKEEKVQELLLRLQGRAENNCLYIEYGINEKITIPITPAFLGQLRSGRSIEKVSFYLGFSIGGPLAYDIEIV

SEQ ID NO: 61 - protein, 233 aa, Homo sapiens
MAATSGTDEPVSGELVSVAHALSLPAESYGNDPDIEMAWAMRAMQHAEVYYKLISSVDPQFLKLTKVDDQIYSEFRKNFETLRIDVLDPEELKSESAKEKWRPFCLKFNGIVEDFNYGTLLRLDCSQGYTEENTIFAPRIQFFAIEIARNREGYNKAVYISVQDKEGEKGVNNGGEKRADSGEEENTKNGGEKGADSGEEKEEGINREDKTDKGGEKGKEADKEINKSGEKAM

SEQ ID NO: 62 - protein, 520 aa, Homo sapiens
MASDPIFTLAPPLHCHYGAFPPNASGWEQPPNASGVSVASAALAASAASRVATSTDPSCSGFAPPDFNHCLKDWDYNGLPVLTTNAIGQWDLVCDLGWQVILEQILFILGFASGYLFLGYPADRFGRRGIVLLTLGLVGPCGVGGAAAGSSTGVMALRFLLGFLLAGVDLGVYLMRLELCDPTQRLRVALAGELVGVGGHFLFLGLALVSKDWRFLQRMITAPCILFLFYGWPGLFLESARWLIVKRQIEEAQSVLRILAERNRPHGQMLGEEAQEALQDLENTCPLPATSSFSFASLLNYRNIWKNLLILGFTNFIAHAIRHCYQPVGGGGSPSDFYLCSLLASGTAALACVFLGVTVDRFGRRGILLLSMTLTGIASLVLLGLWDYLNEAAITTFSVLGLFSSQAAAILSTLLAAEVIPTTVRGRGLGLIMALGALGGLSGPAQRLHMGHGAFLQHVVLAACALLCILSIMLLPETKRKLLPEVLRDGELCRRPSLLRQPPPTRCDHVPLLATPNPAL

SEQ ID NO: 63 - protein, 529 aa, Homo sapiens
MEQEKKLLVSDSNSFMERESLKSPFTGDTSMNNLETVHHNNSKADKLKEKPSEWSKRHRPQHYKHEDAKEMPLTWVQDEIWCHDSYESDGKSENWGNFIAKEEEKPNHQEWDSGEHTNACVQQNSSFVDRPYKCSECWKSFSNSSHLRTHQRTHSGEKPYKCSECAKCFCNSSHLIQHLRMHTGEKPYQCGECGKSFSNTSHLIIHERTHTGEKPYKCPECGKRFSSSSHLIQHHRSHTGEKPYECSVCGKGFSHSYVLIEHQRTHTGEKPYKCPDCGKSFSQSSSLIRHQRTHTGEKPYKCLECEKSFGCNSTLIKHQRIHTGEKPYQCPECGKNFSRSSNLITHQKMHTGEKSYESSEYEESLGQNCNVIEECRIQLGEKPYRCCECGKSFGLSSHLIRHQRTHTGEKPYRCSECWKTFSQSSTLVIHQRTHTGEKPYKCPDCGESFSQSFNLIRHRRTHIGEKPYKCTSCEKCFSRIAYLSQHRKIHVEKPFESPDVGDFPHEWTWKNCSGEMPFISSFSVSNSSS

SEQ ID NO: 64 - protein, 1390 aa, Homo sapiens
MVVLRSSLELHNHSAASATGSLDLSSDFLSLEHIGRRRLRSAGAAQKKPAATTAKAGDGSSVKEVETYHRTRALRSLRKDAQNSSDSSFEKNVEITEQLANGRHFTRQLARQQADKKKEEHREDKVIPVTRSLRARNIVQSTEHLHEDNGDVEVRRSCRIRSRYSGVNQSMLFDKLITNTAEAVLQKMDDMKKMRRQRMRELEDLGVFNETEESNLNMYTRGKQKDIQRTDEETTDNQEGSVESSEEGEDQEHEDDGEDEDDEDDDDDDDDDDDDDDEDDEDEEDGEEENQKRYYLRQRKATVYYQAPLEKPRHQRKPNIFYSGPASPARPRYRLSSAGPRSPYCKRMNRRRHAIHSSDSTSSSSSEDEQHFERRRKRSRNRAINRCLPLNFRKDELKGIYKDRMKIGASLADVDPMQLDSSVRFDSVGGLSNHIAALKEMVVFPLLYPEVFEKFKIQPPRGCLFYGPPGTGKTLVARALANECSQGDKRVAFFMRKGADCLSKWVGESERQLRLLFDQAYQMRPSIIFFDEIDGLAPVRSSRQDQIHSSIVSTLLALMDGLDSRGEIVVIGATNRLDSIDPALRRPGRFDREFLFSLPDKEARKEILKIHTRDWNPKPLDTFLEELAENCVGYCGADIKSICAEAALCALRRRYPQIYTTSEKLQLDLSSINISAKDFEVAMQKMIPASQRAVTSPGQALSTVVKPLLQNTVDKILEALQRVFPHAEFRTNKTLDSDISCPLLESDLAYSDDDVPSVYENGLSQKSSHKAKDNFNFLHLNRNACYQPMSFRPRILIVGEPGFGQGSHLAPAVIHALEKFTVYTLDIPVLFGVSTTSPEETCAQVIREAKRTAPSIVYVPHIHVWWEIVGPTLKATFTTLLQNIPSFAPVLLLATSDKPHSALPEEVQELFIRDYGEIFNVQLPDKEERTKFFEDLILKQAAKPPISKKKAVLQALEVLPVAPPPEPRSLTAEEVKRLEEQEEDTFRELRIFLRNVTHRLAIDKRFRVFTKPVDPDEVPDYVTVIKQPMDLSSVISKIDLHKYLTVKDYLRDIDLICSNALEYNPDRDPGDRLIRHRACALRDTAYAIIKEELDEDFEQLCEEIQESRKKRGCSSSKYAPSYYHVMPKQNSTLVGDKRSDPEQNEKLKTPSTPVACSTPAQLKRKIRKKSNWYLGTIKKRRKISQAKDDSQNAIDHKIESDTEETQDTSVDHNETGNTGESSVEENEKQQNASESKLELRNNSNTCNIENELEDSRKTTACTELRDKIACNGDASSSQIIHISDENEGKEMCVLRMTRARRSQVEQQQLITVEKALAILSQPTPSLVVDHERLKNLLKTVVKKSQNYNIFQLENLYAVISQCIYRHRKDHDKTSLIQKMEQEVENFSCSR

SEQ ID NO: 65 - protein, 309 aa, Homo sapiens
MRQNDKIMCILENRKKRDRKNLCRAINDFQQSFQKPETRREFDLSDPLALKKDLPARQSDNDVRNTISGMQKFMGEDLNFHERKKFQEEQNREWSLQQQREWKNARAEQKCAEALYTETRLQFDETAKHLQKLESTTRKAVCASVKDFNKSQAIESVERKKQEKKQEQEDNLAEITNLLRGDLLSENPQQAASSFGPHRVVPDRWKGMTQEQLEQIRLVQKQQIQEKLRLQEEKRQRDLDWDRRRIQGARATLLFERQQWRRQRDLRRALDSSNLSLAKEQHLQKKYMNEVYTNQPTGDYFTQFNTGSR

SEQ ID NO: 66 - protein, 605 aa, Homo sapiens
MVEGPGCTLNGEKIRARVLPGQAVTGVRGSALRSPQGRALRLAASTVVVSPQAAALNNDSSQNVLSLFNGYVYSGVETLGKELFMYFGPKALRIHFGMKGFIMINPLEYKYKNGASPVLEVQLTKDLICFFDSSVELRNSMESQQRIRMMKELDVCSPEFSFLRAESEVKKQKGRMLGDVLMDQNVLPGVGNIIKNEALFDSGLHPAVKVCQLTDEQIHHLMKMIRDFSILFYRCRKAGLALSKHYKVYKRPNCGQCHCRITVCRFGDNNRMTYFCPHCQKENPQHVDICKLPTRNTIISWTSSRVDHVMDSVARKSEEHWTCVVCTLINKPSSKACDACLTSRPIDSVLKSEENSTVFSHLMKYPCNTFGKPHTEVKINRKTAFGTTTLVLTDFSNKSSTLERKTKQNQILDEEFQNSPPASVCLNDIQHPSKKTTNDITQLSSKVNISPTISSESKLFSPAHKKPKTAHYSSPELKSCNPGYSNSELQINMTDGPRTLNPDSPRCSKHNRLCILRVVRKDGENKGRQFYACPLPREAQCGFFEWADLSFPFCNHGKRSTMKTVLKIGPNNGKNFFVCPLGKEKQCNFFQWAENGPGIKIIPGC

SEQ ID NO: 67 - protein, 207 aa, Homo sapiens
MSEAVRVPSPATPLVVAAAAPEERKGKESEREKLPPIVSAGAGATAGLDRGAKGQISTFSSFISAVSPKKEAAENRSSPAHLVFPNIKNVREPPPICLDVRQKQRTSMDASSSEMKAPVLPEPILPIQPKTVKDFQEDVEKVKSSGDWKAVHDFYLTTFDSFPELNAAFKKDATASFNTIEDSGINAKFVNAVYDTLLNTVSIMTCK

SEQ ID NO: 68 - protein, 154 aa, Homo sapiens
MSRVSGVEACWVRAVRRCCQSWAGQPGRRGGRQSRSVERDEDVGGTDVAEAAGTAGAECLWGAREAFPEAPGGRRGEARVPGEALLHPQPEPGLCLHGPEPTVRVAVGNQLGWPTSPGWPEMFWFWDHSVPGLQPTTVTILCAPCPSSPAASLD

SEQ ID NO: 69 - protein, 234 aa, Homo sapiens
MSVKEGAQRKWAALKEKLGPQDSDPTEANLESADPELCIRLLQMPSVVNYSGLRKRLEGSDGGWMVQFLEQSGLDLLLEALARLSGRGVARISDALLQLTCVSCVRAVMNSRQGIEYILSNQGYVRQLSQALDTSNVMVKKQVFELLAALCIYSPEGHVLTLDALDHYKTVCSQQYRFSIVMNELSGSDNVPYVVTLLSVINAVILGPEDLRARTQLRNEFIGLQLLDVLARLR

SEQ ID NO: 70 - protein, 898 aa, Homo sapiens
MSVTEEDLCHHMKVVVRVRPENTKEKAAGFHKVVHVVDKHILVFDPKQEEVSFFHGKKTTNQNVIKKQNKDLKFVFDAVFDETSTQSEVFEHTTKPILRSFLNGYNCTVLAYGATGAGKTHTMLGSADEPGVMYLTMLHLYKCMDEIKEEKICSTAVSYLEVYNEQIRDLLVNSGPLAVREDTQKGVVVHGLTLHQPKSSEEILHLLDNGNKNRTQHPTDMNATSSRSHAVFQIYLRQQDKTASINQNVRIAKMSLIDLAGSERASTSGAKGTRFVEGTNINRSLLALGNVINALADSKRKNQHIPYRNSKLTRLLKDSLGGNCQTIMIAAVSPSSVFYDDTYNTLKYANRAKDIKSSLKSNVLNVNNHITQYVKICNEQKAEILLLKEKLKAYEEQKAFTNENDQAKLMISNPQEKEIERFQEILNCLFQNREEIRQEYLKLEMLLKENELKSFYQQQCHKQIEMMCSEDKVEKATGKRDHRLAMLKTRRSYLEKRREEELKQFDENTNWLHRVEKEMGLLSQNGHIPKELKKDLHCHHLHLQNKDLKAQIRHMMDLACLQEQQHRQTEAVLNALLPTLRKQYCTLKEAGLSNAAFESDFKEIEHLVERKKVVVWADQTGEQPKQNDLPGISVLMTFSQLGPVQPIPCCSSSGGTNLVKIPTEKRTRRKLMPSPLKGQHTLKSPPSQSVQLNDSLSKELQPIVYTPEDCRKAFQNPSTVTLMKPSSFTTSFQAISSNINSDNCLKMLCEVAIPHNRRKECGQEDLDSTFTICEDIKSSKCKLPEQESLPNDNKDILQRLDPSSFSTKHSMPVPSMVPSYMAMTTAAKRKRKLTSSTSNSSLTADVNSGFAKRVRQDNSSEKHLQENKPTMEHKRNICKINPSMVRKFGRNISKGNLR

SEQ ID NO: 71 - protein, 622 aa, Homo sapiens
MAENTEGDLNSNLLHAPYHTGDPQLDTAIGQWLRWDKNPKTKEQIENLLRNGMNKELRDRLCCRMTFGTAGLRSAMGAGFCYINDLTVIQSTQGMYKYLERCFSDFKQRGFVVGYDTRGQVTSSCSSQRLAKLTAAVLLAKDVPVYLFSRYVPTPFVPYAVQKLKAVAGVMITASHNRKEDNGYKVYWETGAQITSPHDKEILKCIEECVEPWNGSWNDNLVDTSPLKRDPLQDICRRYMEDLKKICFYRELNSKTTLKFVHTSFHGVGHDYVQLAFKVFGFKPPIPVPEQKDPDPDFSTVKCPNPEEGESVLELSLRLAEKENARVVLATDPDADRLAAAELQENGCWKVFTGNELAALFGWWMFDCWKKNKSRNADVKNVYMLATTVSSKILKAIALKEGFHFEETLPGFKWIGSRIIDLLENGKEVLFAFEESIGFLCGTSVLDKDGVSAAVVVAEMASYLETMNITLKQQLVKVYEKYGYHISKTSYFLCYEPPTIKSIFERLRNFDSPKEYPKFCGTFAILHVRDVTTGYDSSQPNKKSVLPVSKNSQMITFTFQNGCVATLRTSGTEPKIKYYAEMCASPDQSDTALLEEELKKLIDALIENFLQPSKNGLIWRSV

SEQ ID NO: 72 - protein, 240 aa, Homo sapiens
MQCFSFIKTMMILFNLLIFLCGAALLAVGIWVSIDGASFLKIFGPLSSSAMQFVNVGYFLIAAGVVVFALGFLGCYGAKTESKCALVTFFFILLLIFIAEVAAAVVALVYTTMAEHFLTLLVVPAIKKDYGSQEDFTQVWNTTMKGLKCCGFTNYTDFEDSPYFKENSAFPPFCCNDNVTNTANETCTKQKAHDQKVEGCFNQLLYDIRTNAVTVGGVAAGIGGLELAAMIVSMYLYCNL

SEQ ID NO: 73 - protein, 1129 aa, Homo sapiens
MPATRKPMRYGHTEGHTEVCFDDSGSFIVTCGSDGDVRIWEDLDDDDPKFINVGEKAYSCALKSGKLVTAVSNNTIQVHTFPEGVPDGILTRFTTNANHVVFNGDGTKIAAGSSDFLVKIVDVMDSSQQKTFRGHDAPVLSLSFDPKDIFLASASCDGSVRVWQISDQTCAISWPLLQKCNDVINAKSICRLAWQPKSGKLLAIPVEKSVKLYRRESWSHQFDLSDNFISQTLNIVTWSPCGQYLAAGSINGLIIVWNVETKDCMERVKHEKGYAICGLAWHPTCGRISYTDAEGNLGLLENVCDPSGKTSSSKVSSRVEKDYNDLFDGDDMSNAGDFLNDNAVEIPSFSKGIINDDEDDEDLMMASGRPRQRSHILEDDENSVDISMLKTGSSLLKEEEEDGQEGSIHNLPLVTSQRPFYDGPMPTPRQKPFQSGSTPLHLTHRFMVWNSIGIIRCYNDEQDNAIDVEFHDTSIHHATHLSNTLNYTIADLSHEAILLACESTDELASKLHCLHFSSWDSSKEWIIDLPQNEDIEAICLGQGWAAAATSALLLRLFTIGGVQKEVFSLAGPVVSMAGHGEQLFIVYHRGTGFDGDQCLGVQLLELGKKKKQILHGDPLPLTRKSYLAWIGFSAEGTPCYVDSEGIVRMLNRGLGNTWTPICNTREHCKGKSDHYWVVGIHENPQQLRCIPCKGSRFPPTLPRPAVAILSFKLPYCQIATEKGQMEEQFWRSVIFHNHLDYLAKNGYEYEESTKNQATKEQQELLMKMLALSCKLEREFRCVELADLMTQNAVNLAIKYASRSRKLILAQKLSELAVEKAAELTATQVEEEEEEEDFRKKLNAGYSNTATEWSQPRFRNQVEEDAEDSGEADDEEKPEIHKPGQNSFSKSTNSSDVSAKSGAVTFSSQGRVNPFKVSASSKEPAMSMNSARSTNILDNMGKSSKKSTALSRTTNNEKSPIIKPLIPKPKPKQASAASYFQKRNSQTNKTEEVKEENLKNVLSETPAICPPQNTENQRPKTGFQMWLEENRSNILSDNPDFSDEADIIKEGMIRFRVLSTEERKVWANKAKGETASEGTEAKKRKRVVDESDETENQEEKAKENLNLSKKQKPLDFSTNQKLSAFAFKQE

SEQ ID NO: 74 - protein, 192 aa, Homo sapiens
MVRAGAVGAHLPASGLDIFGDLKKMNKRQLYYQVLNFAMIVSSALMIWKGLIVLTGSESPIVVVLSGSMEPAFHRGDLLFLTNFREDPIRAGEIVVFKVEGRDIPIVHRVIKVHEKDNGDIKFLTKGDNNEVDDRGLYKEGQNWLEKKDVVGRARGFLPYVGMVTIIMNDYPKFKYALLAVMGAYVLLKRES

SEQ ID NO: 75 - protein, 247 aa, Homo sapiens
MNPLLILTFVAAALAAPFDDDDKIVGGYNCEENSVPYQVSLNSGYHFCGGSLINEQWVVSAGHCYKSRIQVRLGEHNIEVLEGNEQFINAAKIIRHPQYDRKTLNNDIMLIKLSSRAVINARVSTISLPTAPPATGTKCLISGWGNTASSGADYPDELQCLDAPVLSQAKCEASYPGKITSNMFCVGFLEGGKDSCQGDSGGPVVCNGQLQGVVSWGDGCAQKNKPGVYTKVYNYVKWIKNTIAANS

SEQ ID NO: 76 - protein, 128 aa, Homo sapiens
MESKEERALNNLIVENVNQENDEKDEKEQVANKGEPLALPLNVSEYCVPRGNRRRFRVRQPILQYRWDIMHRLGEPQARMREENMERIGEEVRQLMEKLREKQLSHSLRAVSTDPPHHDHHDEFCLMP

SEQ ID NO: 77 - protein, 545 aa, Homo sapiens
PRCAGRSSPVRKRHGGRRAGGKDTLVSVPRSVQDSGQGGREKLELVLSNLQADVLELLLEFVYTGSLVIDSANAKTLLEAASKFQFHTFCKVCVSFLEKQLTASNCLGVLAMAEAMQCSELYHMAKAFALQIFPEVAAQEEILSISKDDFIAYVSNDSLNTKAEELVYETVIKWIKKDPATRTQYAAELLAVVRLPFIHPSYLLNVVDNEELIKSSEACRDLVNEAKRYHMLPHARQEMQTPRTRPRLSAGVAEVIVLVGGRQMVGMTQRSLVAVTCWNPQNNKWYPLASLPFYDREFFSVVSAGDNIYLSGGMESGVTLADVWCYMSLLDNWNLVSRMTVPRCRHNSLVYDGKIYTLGGLGVAGNVDHVERYDTITNQWEAVAPLPKAVHSAAATVCGGKIYVFGGVNEAGRAAGVLQSYVPQTNTWSFIESPMIDNKYAPAVTLNGFVFILGGAYARATTIYDPEKGNIKAGPNMNHSRQFCSAVVLDGKIYATGGIVSSEGPALGNMEAYEPTTNTWTLLPHMPCPVFRHGCVVIKKYIQSG